(19) United States
(12) Patent Application Publication
(10) Pub. No.: US 2011/0152112 A1
(43) Pub. Date: Jun. 23, 2011

(54) DIAGNOSIS AND TREATMENT OF REVERSE CHOLESTEROL TRANSPORT DEFICIENCY-RELATED DISEASES

(75) Inventor: Jan O. Johansson, Portola Valley, CA (US)

(73) Assignee: Artery Therapeutics, Inc., Portola Valley, CA (US)

(21) Appl. No.: 12/976,024

(22) Filed: Dec. 22, 2010

Related U.S. Application Data
(60) Provisional application No. 61/289,989, filed on Dec. 23, 2009.

(51) Int. Cl. C40B 30/04; C40B 30/06; C12Q 1/02

(52) U.S. Cl. 506/9; 506/10; 435/29

(57) ABSTRACT

The present invention provides compositions and methods to assess the state of a reverse cholesterol transport (RCT). In one aspect of the invention provides methods, compositions and kits for diagnosing a subject with deficient reverse cholesterol transport (RCT). In another aspect, the present invention provides a method of identifying responders to RCT treatment. In yet another aspect, the present invention provides a method of treating a subject with RCT deficiency. Also provided by the present invention is a method for drug screening and/or assessing risk of toxicity associated with RCT treatments.

### CROSS-REFERENCE

This application claims the benefit of U.S. Provisional Application No. 61/289,989, filed Dec. 23, 2009, which application is incorporated herein by reference.

### BACKGROUND OF THE INVENTION

Atherosclerosis and risk of developing cardiovascular disease (CVD) is dependent on lipid and macrophage retention in the vascular wall. The cholesterol pool in the macrophage is a key factor for the build up of atherosclerosis and CVD risk. The macrophage cholesterol pool is governed by the influx and efflux of cholesterol and other lipids. This lipid flux is regulated by specific receptors and transporters on the cell surface, and notably various apolipoproteins are implemented in this process. Apolipoproteins are large, characterized by several structures (alpha helices, beta shields etc.) and they have multiple functions. Apolipoproteins circulate in the body almost exclusively in complexes with lipids and other (apo) proteins. Little is known about the detailed mechanism by which cholesterol and macrophage retention in the vascular wall (and CVD) is regulated.

In view of the potential capacity of reverse cholesterol transport (RCT) and Reverse Lipid Transport (RLT, including transport of other lipids than cholesterol)) to treat CVD, there is a medical need to improve diagnostic means to identify subjects with RCT deficiency and to develop methods for assessing those individuals that would be responders to treatment as opposed to those that would not respond to treatment.

### SUMMARY OF THE INVENTION

The invention provides methods, compositions and kits for the determination of activity of a reverse cholesterol transport (RCT) pathway. For convenience purposes only the invention will be described in terms of reverse RCT pathways and transporters. However, the invention encompasses RLT pathways and transporter. Thus, in the contest of the methods, compositions and kits described herein the terms RTC and RLT can be used interchangeably.

In some embodiments, the invention provides methods of prognosing, diagnosing, and/or predicting a response to treatment of a condition associated with a deficiency in a reverse cholesterol transport (RCT) pathway in a subject comprising the steps of: (a) providing a population of cells from the subject; (b) contacting the population of cells with a modulator that specifically modulates a reverse cholesterol transporter pathway; (c) assessing lipid efflux profile, mRNA expression, protein expression, protein activation level and/or a phenotype in the at least one cell treated with the modulator or a medium comprising said cell; (d) determining whether there is a deficiency in the reverse cholesterol transport pathway of the subject, where the determining is based in the assessing of lipid efflux profile, mRNA expression, protein expression, protein activation level and/or the phenotype in the at least cell; and (e) prognosing, diagnosing, and/or predicting a response to treatment of the condition associated with a deficiency in a RCT pathway, where the prognosing, diagnosing, and/or predicting a response to treatment is based in the determining in step (d). In some embodiments, the cell is a macrophage or a macrophage-like cell. The medium comprising the cell can be, for example, tissue, organ, blood, serum, plasma, body fluid, or culture media.

In some embodiments, the invention provides methods of screening of compounds for treatment of a condition associated with reverse cholesterol transport deficiency and/or assessing risk of toxicity of a treatment of a condition associated with reverse cholesterol transport deficiency comprising the steps of: (a) providing a macrophage or a macrophage-like cell; (b) contacting the macrophage or macrophage-like cell with one or more compounds, where the one or more compounds are possible candidates for the treatment of a condition associated with reverse cholesterol transport deficiency, and/or where the one or more compounds are used in the treatment of a condition associated with reverse cholesterol transport deficiency; (c) assessing lipid efflux profile, mRNA expression, protein expression, protein activation level and/or a phenotype in the macrophage or macrophage-like cell treated with the compound or a medium comprising the cell; and (d) selecting the one or more compounds for treatment of the condition associated with reverse cholesterol transport deficiency and/or determining toxicity of a treatment of the condition associated with reverse cholesterol transport deficiency, where the selecting and/or the determining are based in the assessing from step (c). The medium comprising the cell can be, for example, tissue, organ, blood, serum, plasma, body fluid, or culture media.

In some embodiments, the invention provides methods comprising prognosing, diagnosing, and/or predicting a response to treatment of a condition associated with a deficiency in a reverse cholesterol transport (RCT) pathway in a subject the methods comprising the steps of (a) administering a subject with a modulator that specifically modulates a reverse cholesterol transporter pathway; (b) assessing lipid efflux profile, mRNA expression, protein expression, protein activation level and/or a phenotype in the at least one cell from the subject or a medium comprising the cell; (c) determining whether there is a deficiency in the reverse cholesterol transport pathway of the subject, where the determining is based in the assessing of lipid efflux profile, mRNA expression, protein expression, protein activation level and/or the phenotype in the at least one cell; and (d) prognosing, diagnosing, and/or predicting a response to treatment of the condition associated with a deficiency in a reverse cholesterol transport (RCT) pathway, where the prognosing, diagnosing, and/or predicting a response to treatment is based in the determining in step (c). In some embodiments, the cell is a macrophage or a macrophage-like cell. The medium comprising the cell can be, for example, tissue, organ, blood, serum, plasma, body fluid, or culture media.

In some embodiments, assessing lipid efflux profile includes measuring total Cholesterol, cholesterol ester, HDL, LDL, IDL, VLDL, triglycerides ratio and phospholipids selected from the group consisting of sphingolipids and phosphatidyl choline. In some embodiments, the sphingolipids are selected from the group consisting of spingosines, ceramides and sphoingomyelings. Thus, in some embodiments, assessing lipid efflux profile includes measuring cholesterol ester, spingosines, ceramides, sphoingomyelings and phosphatidyl choline. In some embodiments, assessing lipid efflux profile includes measuring the conversion of α-mobility HDL particles to pre-β1-HDL. Thus in some embodiments, assessing lipid efflux profile includes measuring cholesterol ester, spingosines, ceramides, sphoingomyelings, phosphatidyl choline, and measuring the conversion of α-mobility HDL particles to pre-β1-HDL.

In some embodiments, the invention provides methods for determining a RCT pathway state by protein expression in response to at least one RCT pathway modulator in the at least one cell. In some embodiments, the protein is an inflammatory protein. In some embodiments, the proteins are selected from the group consisting of CRP, Fibrinogen, Haptoglobin, IL-18, SAP (serum amyloid P component), Rantes, TIMP-1, VCAM-1, MIP-1beta, MPO, VEGF-alpha and IL-7.

In some embodiments, the methods of the invention may further comprise comparing the lipid efflux profile, mRNA expression, protein expression, and/or protein activation level to a predetermined threshold value.

In some embodiments, the invention provides methods for prognosing, diagnosing, and/or predicting a response to treatment of a condition associated with a deficiency in a reverse cholesterol transport (RCT) pathway in a subject the method comprising the steps of: (a) administering a subject with a modulator that specifically modulates a reverse cholesterol transporter pathway; (b) assessing the mobilization of a biomarker from tissue to plasma in the subject; and (c) prognosing, diagnosing, and/or predicting a response to treatment of the condition associated with a deficiency in a reverse cholesterol transport (RCT) pathway, where the prognosing, diagnosing, and/or predicting a response to treatment is based in the assessing in step (b).

In some embodiments, the present invention provides a method of prognosing and/or diagnosing a subject with deficiency in the RCT pathway, comprising: (a) isolating macrophage or a macrophage-like cell from the subject; (b) contacting the macrophage or macrophage-like cell with a compound that specifically modulates a reverse cholesterol transporter pathway; (c) assessing lipid efflux profile of the macrophage or macrophage-like cell treated with the compound as compared to lipid efflux profile of a control cell of the same type; and (d) determining whether there is a deficiency in the reverse cholesterol transport pathway of the subject. The medium comprising the cell can be, for example, tissue, organ, blood, serum, plasma, body fluid, or culture media.

In some embodiments, the present invention provides a method of predicting or identifying response of a subject with deficiency in reverse cholesterol transport (RCT) to treatment with a modulator of a reverse cholesterol transport pathway comprising: (a) isolating macrophage or a macrophage-like cell from the subject; (b) contacting the macrophage or macrophage-like cell with the modulator that is specific for a reverse cholesterol transporter pathway; (c) comparing lipid efflux profiles of the macrophage or macrophage-like cell treated with or without the modulator; and (d) determining whether the subject responds to treatment with the modulator. In some embodiments, the subject responds to the treatment with the modulator if there is a change in lipid efflux profile as compared to that of a control cell.

In some embodiments, the present invention provides a method of treating a RCT related disease comprising administering to a subject in need thereof an effective amount of a modulator that is specific for a reverse cholesterol transporter.

In some embodiments, the present invention provides a method for assessing risk of toxicity associated with treatment of reverse cholesterol transport deficiency comprising: (a) isolating macrophage or a macrophage-like cell from the subject; (b) contacting the macrophage or macrophage-like cell with the modulator that is specific for a reverse cholesterol transporter pathway; (c) assessing lipid efflux profile of the macrophage or macrophage-like cell treated with the modulator; and (d) determining toxicity on the subject associated with treatment of the modulator.

By using modulators (e.g. peptides) with selective effects in the key transporters, receptors and or proteins associated with RCT deficiencies in efflux can be assessed. By assessing mRNA changes, for example, in cholesterol transporting proteins cells can be characterized for RCT properties. In some embodiments, by designing peptides with selective effects on a reverse cholesterol transporter, for example the ABCA1 transporter, ABCG1 transporter, other transporters and scavenger receptor B1, the capacity and deficiency of these RCT pathways can be diagnosed functionally, in various cells including but not limited to macrophages, foam cells, in vitro and in vivo. In some embodiments, the compositions and methods described herein can be used for testing dose and time response for peptide mediated efflux of free cholesterol and various phospholipids to create a tool for pharmacokinetic assessment in drug development and development of a biomarker. For example, specific increase in plasma concentration of a lipid can be assessed at various time points to assess a treatment capacity to mobilize lipid from tissue to plasma.

In some embodiments, the subject methods are used to diagnosing and/or identifying patients with a) reverse cholesterol transport deficiency, and b) responders vs. non-responders to treatment of an RCT deficient related condition. In some embodiments, the lipid efflux profile includes but is not limited to cholesterol and phospholipid efflux profile from 1) different macrophage cell types or macrophage-like cells, 2) intact artery tissues, and 3) in vitro mobilization to plasma.

The methods of the present invention can be applied at a cell level, an organ level (for example lipid removal from an arterial segment) and in plasma by assessing mobilization (increase in concentrations) of lipids from peripheral tissue to plasma.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows attenuation of TNFa-induced VCAM adhesion to HUVEC.

FIG. 2 shows acceptor-mediated cholesterol efflux from RAW-macrophages.

FIG. 3 shows Acceptor-mediated cholesterol efflux from RAW-macrophages.

FIG. 4 shows that all active agents lowered cholesterol ester content of carotid artery by ˜20%.

FIG. 5 shows that all active agents lowered sphingosine and ceramide content of carotid artery by 20 to 40%.

FIG. 6 shows that all active agents lowered sphingomyelin content of carotid artery by 10 to 30%.

FIG. 7 shows serum cholesterol levels following treatment with vehicle, D4F peptide, AT5261 free, and AT5261/PL-complex peptides.

FIG. 8 shows serum triglyceride levels following treatment with vehicle, D4F peptide, AT5261 free, and AT5261/PL-complex peptides.

FIG. 9 shows body weight change over the duration of treatment with vehicle, D4F peptide, AT5261 free, and AT5261/PL-complex peptides.

FIG. 10 shows plasma lipid concentrations in mice following treatment with vehicle, D4F peptide, AT5261 free, and AT5261/PL-complex peptides.

FIG. 11 shows that AT5261 Peptide Converts α-mobility HDL particles to preβ1-HDL.

FIG. 12 shows the lipid efflux responses to peptide ATI-5261 and ApoA-I in transformed macrophage cell-line, J774 (mouse).

FIG. 13 shows the lipid efflux responses to peptide ATI-5261 and ApoA-I in transformed macrophage cell-line, J774 (mouse).

FIG. 14 shows that AT5261 Peptide Converts α-mobility HDL particles to preβ1-HDL.

### DETAILED DESCRIPTION OF THE INVENTION

Reference will now be made in detail to particularly preferred embodiments of the invention. Examples of the preferred embodiments are illustrated in the following Examples section.

Unless defined otherwise, all technical and scientific terms used herein have the same meaning as is commonly understood by one of skill in the art to which this invention belongs. As used in this specification and the appended claims, the singular forms “a”, “an”, and “the” include plural references unless the context clearly dictates otherwise. All patents and publications referred to herein are incorporated by reference in their entirety.

General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Introduction:

The present invention provides methods, compositions and kits for prognosing, diagnosing, screening compounds and/or predicting a response to treatment of a condition associated with a deficiency in a reverse cholesterol transport (RCT) pathway. RCT pathway deficiencies play a role in the development of cardiovascular diseases such as artherosclerosis. RCT is considered a key anti-atherogenic and anti-atherosclerotic process, and is generally believed to be the explanation for anti-atherogenic and anti-atherosclerotic properties as well as the clinical correlation with reduced cardiovascular risk of the high density lipoprotein (HDL) fraction of plasma.

Due to the growing global impact of cholesterol-related disease, and due to the complexities of cholesterol metabolism and its study, an in vivo method for measuring the rate of RCT is needed and would have great utility for medical care and drug discovery and development. The identification, selection, evaluation and development (e.g., clinical or pre-clinical dose finding and optimization of dosages, measurement of efficacy) of candidate therapies, the diagnosis of cholesterol-related disease, the management of subjects, including evaluation of disease progression or response to therapy, and the design and testing of medical devices or tools for use in cholesterol-related disease management, diagnosis or treatment are all processes which would benefit from the practice of the methods of the present invention. Evaluating subjects prior to enrollment in clinical trials is one beneficial use of the present invention. Evaluating subjects in order to predict whether or not they will respond to a candidate therapy is another beneficial use of the present invention. The invention has further uses directed toward the development of disease criteria (i.e., a combination of risk factors that indicate a disease or pre-disease state) that can be used to classify subjects and recommend treatment.

The methods are generally carried out in mammalian subjects, including humans. Mammals include, but are not limited to, primates, farm animals, sport animals, pets such as cats and dogs, guinea pigs, rabbits, hamsters, mice, rats, humans and the like.

In some embodiment, the methods, compositions and kits of the invention can be used to identify RCT deficiency, identify responders to receptor activation, identify responders to a certain treatment, assess treatment progress and/or predict treatment outcome. In some embodiments, the invention provides methods and compositions for the screening of compounds for treatment of a condition associated with RCT deficiency and/or assessing risk of toxicity of a treatment of a condition associated with RCT deficiency. In some embodiments, the invention provides methods, compositions and kits to identify new druggable targets for the treatment of a condition associated with RCT deficiency.

In some embodiments the methods of the invention comprise the use a modulator specific for a RCT pathway. For example, a cell membrane has different lipids at the inner and outer leaflet. These lipids have both structural (e.g., building the cell membrane wall) and functional properties (e.g., receptor signaling into the cell, inflammatory mediators into plasma). Thus, how a modulator specific for a component of a RCT pathway or specific manipulation affects the specific lipid removal (e.g., inner and/or outer leaflet, specific lipids) will have impact on fundamental biology of importance for disease states and can be used to, for example, prognose, diagnose, predict a response to treatment of a condition associated with a deficiency in a reverse cholesterol transport (RCT) pathway, drug screening and/or assessing risk of toxicity of a treatment.

Reverse Cholesterol Transport

Cholesterol is a lipid found in the cell membranes and transported in the blood plasma of all animals. It is an essential component of mammalian cell membranes where it is required to establish proper membrane permeability and fluidity. Cholesterol is the principal sterol synthesized by animals while smaller quantities are synthesized in other eukaryotes such as plants and fungi. In contrast cholesterol is almost completely absent among prokaryotes. Most cholesterol is synthesized by the body but significant quantities can also be absorbed from the diet. While minimum level of cholesterol is essential for life, excess can contribute to diseases such as atherosclerosis.

Since cholesterol is insoluble in blood, it is transported in the circulatory system within lipoproteins, complex spherical particles which have an exterior composed mainly of water-soluble proteins; fats and cholesterol are carried internally. There is a large range of lipoproteins within blood, generally called, from larger to smaller size: chylomicrons, very low density lipoprotein (VLDL), intermediate density lipoprotein (IDL), low density lipoprotein (LDL) and high density lipoprotein (HDL). The cholesterol within all the various lipoproteins is identical. Cholesterol is minimally soluble in water; it cannot dissolve and travel in the water-based bloodstream. Instead, it is transported in the bloodstream by lipoproteins that are water-soluble and carry cholesterol and triglycerides internally. The apolipoproteins forming the surface of the given lipoprotein particle determine from what cells cholesterol will be removed and to where it will be supplied.

Cholesterol is transported towards peripheral tissues by the lipoproteins chylomicrons, very low density lipoproteins (VLDL) and low-density lipoproteins (LDL). Large numbers of small dense LDL (sdLDL) particles are strongly associated with the presence of atheromatous disease within the arteries. For this reason, LDL is referred to as “bad cholesterol”. On the other hand, high-density lipoprotein (HDL) particles transport cholesterol back to the liver for excretion. In contrast, having small numbers of large HDL particles is independently associated with atheromatous disease progression within the arteries.

Chylomicrons: Chylomicrons are the largest (1000 nm) and least dense (<0.95) of the lipoproteins. They contain only 1-2% protein, 85-88% triglycerides, ˜8% phospholipids, ˜3% cholesteryl esters and ˜1% cholesterol. Chylomicrons contain several types of apolipoproteins including apo-AI, II & IV, apo-B48, apo-CI, II & III, apo-E and apo-H. Chylomicrons are produced for the purpose of transporting dietary triglycerides and cholesterol absorbed by intestinal epithelia. Chylomicron assembly originates in the intestinal mucosa. Excretion into the plasma is facilitated through the lymphatic system. In the plasma, chylomicrons acquire apo-CII and apo-E from HDL. Once transported to tissues, triglycerides contained in chylomicrons are hydrolyzed by apo-CII-dependent activation of lipoprotein lipase contained on the endothelial cell walls. The chylomicron remnant, including residual cholesterol, is taken up by the liver via receptor-mediated endocytosis by recognition of its apo-E component.

Very Low Density Lipoproteins (VLDL) Very low density lipoproteins are the next step down from chylomicrons in terms of size and lipid content. They are approximately 25-90 nm in size (MW 6-27 million), with a density of ˜0.98. They contain 5-12% protein, 50-55% triglycerides, 18-20% phospholipids, 12-15% cholesteryl esters and 8-10% cholesterol. VLDL also contains several types of apolipoproteins including apo-B100, apo-CI, II & III and apo-E. VLDL also obtains apo-CII and apo-E from plasma HDL. VLDL assembly in the liver involves the early association of lipids with apo-B100 mediated by microsomal triglyceride transfer protein while apo-B100 is translocated to the lumen of the ER. Lipoprotein lipase also removes triglycerides from VLDL in the same way as from chylomicrons.

Intermediate Density Lipoproteins (IDL) Intermediate density lipoproteins are smaller than VLDL (40 nm) and more dense (˜1.0). They contain the same apolipoproteins as VLDL. They are composed of 10-12% protein, 24-30% triglycerides, 25-27% phospholipids, 32-35% cholesteryl esters and 8-10% cholesterol. IDLs are derived from triglyceride depletion of VLDL. IDLs can be taken up by the liver for reprocessing, or upon further triglyceride depletion, become LDL.

Low Density Lipoproteins (LDL) and Lipoprotein (a) Low density lipoproteins are smaller than IDL (26 nm) (MW approximately 3.5 million) and more dense (˜1.04). They contain the apolipoprotein apo-B100. LDL contains 20-22% protein, 10-15% triglycerides, 20-28% phospholipids, 37-48% cholesteryl esters and 8-10% cholesterol. LDL and HDL transport both dietary and endogenous cholesterol in the plasma. LDL is the main transporter of cholesterol and cholesteryl esters and makes up more than half of the total lipoprotein in plasma. LDL is absorbed by the liver and other tissues via receptor mediated endocytosis. The cytoplasmic domain of the LDL receptor facilitates the formation of coated pits; receptor-rich regions of the membrane. The ligand binding domain of the receptor recognizes apo-B100 on LDL, resulting in the formation of a clathrin-coated vesicle. ATP-dependent proton pumps lower the pH inside the vesicle resulting dissociation of LDL from its receptor. After loss of the clathrin coat the vesicles fuse with lysozomes, resulting in peptide and cholesteryl ester enzymatic hydrolysis. The LDL receptor can be recycled to the cell membrane. Insulin, tri-iodothyronine and dexamethasome have shown to be involved with the regulation of LDL receptor mediated uptake.

High Density Lipoproteins High density lipoproteins are the smallest of the lipoproteins (6-12.5 nm) (MW 175-500 KD) and most dense (˜1.12). HDL contains several types of apolipoproteins including apo-AI, II & IV, apo-CI, II & III, apo-D and apo-E. HDL contains approximately 55% protein, 3-15% triglycerides, 26-46% phospholipids, 15-30% cholesteryl esters and 2-10% cholesterol. HDL is produced as a protein rich particle in the liver and intestine, and serves as a circulating source of Apo-CI & II and Apo-E proteins. The HDL protein particle accumulates cholesteryl esters by the esterification of cholesterol by lecithin-cholesterol acyl-transferase (LCAT). LCAT is activated by apo-AI on HDL. HDL can acquire cholesterol from cell membranes and can transfer cholesteryl esters to VLDL and LDL via transferase activity in apo-D. HDL can return to the liver where cholesterol is removed by reverse cholesterol transport, thus serving as a scavenger to free cholesterol. The liver can then excrete excess cholesterol in the form of bile acids. In a normal fasting individual, HDL concentrations range from 1.0-2.0 g/L.

Lipid Transport-ATP mediated transporter Reverse cholesterol transport is a multi-step process resulting in the net movement of cholesterol from peripheral tissues back to the liver via the plasma compartment. Cellular cholesterol efflux is mediated by HDL, acting in conjunction with the cholesterol esterifying enzyme, lecithin: cholesterol acyltransferase. Cholesteryl ester accumulating in HDL can then follow a number of different fates: uptake in the liver in HDL containing apolipoprotein (particle uptake) by LDL receptors, selective uptake of HDL cholesteryl ester in liver or other tissues involving scavenger receptor B1, or transfer to triglyceride-rich lipoproteins as a result of the activity of cholesteryl ester transfer protein, with subsequent uptake of triglyceride-rich lipoprotein remnants in the liver. Recently, several groups have taken a molecular approach to analyzing the different components of reverse cholesterol transport, by over- or under-expression of individual molecules in induced mutant mouse models, or by the study of human mutations involving molecules of reverse cholesterol transport. Such studies reveal that over-expression of the major HDL apoprotein, apolipoprotein A-I, is clearly anti-atherogenic. However, over- or under-expression of molecules such as cholesteryl ester transfer protein, which have opposite effects on HDL levels and reverse cholesterol transport, suggest that both HDL levels as well as the dynamics of cholesterol movement through HDL are involved in the anti-atherogenic actions of HDL (Tall AR Eur Heart J. 1998 February; 19 Suppl A:A31-5).

In some embodiments, the present invention provides methods, compositions and kits for prognosing, diagnosing, and/or predicting a response to treatment of a condition associated with a deficiency in a reverse cholesterol transport (RCT) pathway by contacting a cell with a modulator that is specific for a reverse cholesterol transporter. In some embodiments, the invention provides methods, compositions and kits for the screening of compounds for treatment of a condition associated with RCT deficiency and/or assessing risk of toxicity of a treatment of a condition associated with RCT deficiency by contacting a cell with a modulator that is specific for a reverse cholesterol transporter. In some embodiments, the invention provides methods, compositions and kits to identify new druggable targets for the treatment of a condition associated with RCT deficiency by contacting a cell with a modulator that is specific for a reverse cholesterol transporter. In some embodiments, the cell is a macrophage or macrophage like cell. In some embodiments, the present invention provides a method of treating a (RCT) related disease comprising administering to a subject in need thereof an effective amount of a modulator that is specific for a reverse cholesterol transporter. In some embodiments, following the treatment the invention provides methods for predicting a response to treatment by contacting a cell that has been subjected to a treatment with a modulator that is specific for a reverse cholesterol transporter.

In some embodiments, the reverse cholesterol transporter is ATP-binding cassette transporter. ATP-binding cassette transporters (ABC-transporter) are members of a superfamily, i.e. ATP-mediated transporter family that is one of the largest and most ancient families with representatives in all extant phyla from prokaryotes to humans. These are transmembrane proteins that function in the transport of a wide variety of substrates across extra- and intracellular membranes, including metabolic products, lipids and sterols, and drugs. Proteins are classified as ABC transporters based on the sequence and organization of their ATP-binding domain(s), also known as nucleotide-binding folds (NBFs). ABC transporters are involved in tumor resistance, cystic fibrosis, bacterial multidrug resistance, and a range of other inherited human diseases.

ABC-transporters utilize the energy of ATP hydrolysis to transport various substrates across cellular membranes. Within eukaryotes, ABC-transporters mainly transport molecules to the outside of the plasma membrane or into membrane-bound organelles such as the endoplasmic reticulum, mitochondria, etc. The transported compounds include but are not limited to lipids and sterols; ions and small molecules; drugs and large polypeptides.

In some embodiments, the reverse cholesterol transporter is ATP-binding cassette, sub-family A member 1 (ABCA1). The ABCA1 gene belongs to a group of genes called the ATP-binding cassette family, which provides instructions for making proteins that transport molecules across cell membranes. This transporter is a major regulator of cellular cholesterol and phospholipid homeostasis. With cholesterol as its substrate, this protein functions as a cholesterol efflux pump in the cellular lipid removal pathway. Mutations in this gene have been associated with Tangier's disease and familial high-density lipoprotein deficiency. The ABCA1 protein is produced in many tissues, but especially in the liver and macrophages. ABCA1 transfers cholesterol and phospholipids across the cell membrane to the outside of the cell. These substances are then taken up by apolipoprotein A1 (apoA1) that circulates in the bloodstream. More specifically, ABCA1 exports excess cellular cholesterol to apoA1 associated with nascent-high-density lipoprotein (HDL) discs, which are assembled in hepatocytes and released into circulation. ApoA1 is used to make HDL. HDL particles carry cholesterol from the body's tissues to the liver for elimination through bile, a yellow substance made by the liver that aids in the digestion of fats. Mature HDL particles are internalized by hepatocytes and free cholesterol is released concomitantly. Free oxysterol and cholesterol levels in hepatocytes provide feedback regulation to cholesterol and fatty acid biosynthesis. The process of removing excess cholesterol from the cells and transporting it to the liver for removal is extremely important for the homeostasis of cholesterol and the cardiovascular health. There is a wide consensus that cholesterol and/or cholesteryl ester accumulation in macrophages plays a role in atherogenesis and that this process occurs through an inflammatory process (Ross, R. 1999. N Engl. J. Med. 340:115-126). A corollary to this premise is that factors that affect the balance between cholesterol retention and cholesterol efflux in macrophages will be pro- or antiatherogenic. With ABCA1 deficiency, apoA-I is rapidly cleared before it is able to acquire cholesterol (Bojanovski, D., et. al. 1987. J. Clin. Invest. 80:1742-1747.). Thus, the loss of HDL in ABCA1 deficiency ay account for the severe cholesteryl ester storage phenotype seen in tissue macrophages and in hepatocytes of Tangier patients and WHAM chickens.

ABCA1 is well documented as the gate keeper for reverse cholesterol transport (Alan D. Attie, et. al. Journal of Lipid Research, Vol. 42, 1717-1726, November 2001). Extrahepatic tissues synthesize cholesterol and also derive cholesterol through the uptake of lipoproteins via the LDL receptor and scavenger receptors. The cholesteryl ester is in a dynamic equilibrium with free cholesterol, through the opposing actions of acylCoA:cholesterol acyltransferase (ACAT) and neutral cholesterol esterase. Free cholesterol effluxes to extracellular acceptors, most notably phospholipid/apoA-I disks (pre-β-HDL). This process is directly (or indirectly through phospholipid efflux) dependent on functional ABCA1. Proper lipidation is essential for the stability of HDL. In the absence of sufficient cholesterol efflux, apoA-I is rapidly cleared from the circulation by the kidneys. Cholesterol that associates with apoA-1/phospholipid disks is a substrate for lecithin:cholesterol acyltransferase (LCAT). LCAT transfers a fatty acyl chain from phosphatidylcholine to cholesterol, forming cholesteryl ester. The cholesteryl ester partitions into the hydrophobic core of the lipoprotein, thus forming spherical HDL particles. These particles can then deliver cholesteryl ester to the liver and steroidogenic tissues. B: Selective uptake of cholesteryl esters from HDL. The interaction of spherical HDL particles with the scavenger receptor class B type I (SR-BI) leads to selective delivery of cholesteryl esters. SR-BI interacts with spherical HDL particles but not with apoA-I or poorly lipidated HDL disks. The cholesteryl esters are hydrolyzed by a neutral cholesterol esterase, providing free cholesterol for secretion across the apical (bile canalicular) membrane of the hepatocyte and for bile acid synthesis. Although the diagram shows cholesterol coming from extrahepatic tissues, growing evidence suggests that a major source of cholesterol for ABCA1-mediated transport to HDL is the liver.

In some embodiments, the reverse cholesterol transporter is ATP-binding cassette, sub-family G member 1 (ABCG1). ABCG1 is another cholesterol transporter. Studies indicate a synergistic relationship between ABCA1 and ABCG1 in peripheral tissues, where ABCA1 lipidates any lipid-poor/free apoA-I to generate nascent or pre-β-HDL. These particles in turn may serve as substrates for ABCG1-mediated cholesterol export (Ingrid C. Gelissen et. al. Arteriosclerosis, Thrombosis, and Vascular Biology. 2006; 26:534).

In practicing the subject methods disclosed herein, in some embodiments, RCT deficiency is determined by measuring efflux and/or plasma concentrations of lipid, sterol, cholesterol, triglyceride, phospholipid or a tocopherol molecule. In some embodiments, RCT deficiency is determined by contacting a cell with a RCT transporter modulator where RCT transporter is an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

Reverse Cholesterol Transport Modulator

The invention provides for reverse cholesterol transport modulators. The reverse cholesterol transport modulators are particularly to determine RCT pathway deficiencies.

In some embodiments, the reverse cholesterol transport modulator is a small molecule, DNA, RNA, an aptamer, a peptide or a nucleotide. In some embodiments, the reverse cholesterol transport modulator can bind to a phospholipid surface such as cell membranes or complexes of proteins and phospholipids (e.g. lipoproteins). In some embodiments, the reverse cholesterol transport modulator is a hydrophobic molecule that binds a phospholipid surface. In some embodiments, the reverse cholesterol transport modulator is a hydrophobic molecule that binds to a phospholipid surface selected from the group consisting of chylomicrons, HDL, LDL and VLDL. In some embodiments, the reverse cholesterol transport modulator is a hydrophobic molecule that binds to HDL. In some embodiments, the reverse cholesterol transport modulator is selected from the group consisting of lipid, phospholipid, fat, protein, peptide, amino acid, organic molecule, small molecule, DNA, RNA, aptamers, peptides and carbohydrates. In some embodiments, the reverse cholesterol transport modulator is a peptide. Thus, in some embodiments, the reverse cholesterol transport modulator is a hydrophobic molecule that binds to HDL and is selected from the group consisting of lipid, phospholipid, fat, protein, peptide, amino acid, organic molecules, small molecule, DNA, RNA, aptamers, peptides and carbohydrates. In some embodiments, reverse cholesterol transport modulator is a peptide. In some embodiments, the reverse cholesterol transport modulator is a hydrophobic molecule that binds to a lipoprotein. In some embodiments, the reverse cholesterol transport modulator is a naturally occurring peptide. In some embodiments, the reverse cholesterol transport modulator is a peptide mimetic. In some embodiments, the reverse cholesterol transport modulator is a peptide comprising natural amino acids. In some embodiments, the reverse cholesterol transport modulator is a peptide comprising non-naturally occurring amino acids. In some embodiments, the reverse cholesterol transport modulator is a peptide with a reversed sequence, where the N terminal and C terminal thereof are reversed. In some embodiments, the reverse cholesterol transport modulator is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

A. Peptides and/or Peptide Mimetics

In some embodiments, the reverse cholesterol transport modulator is a peptide or a peptide mimetic. In some embodiments, the peptide or a peptide mimetic is an amphitropic protein. In some embodiments, the amphitropic proteins associate with phospholipid surfaces via a hydrophobic anchor structure. Examples of hydrophobic anchor structures include, but are not limited to, amphipathic α-helixes, exposed nonpolar loops, post-translationally acylated or lipidated amino acid residues, or acyl chains of specifically bound regulatory lipids such as phosphatidylinositol phosphates. In some embodiments, the peptide or peptide mimetic comprises at least one amphipathic alpha-helix. In some embodiments, the peptide or peptide mimetic comprises at least one exposed nonpolar loops. In some embodiments the peptide or peptide mimetic comprises at least one post-translationally acylated or lipidated amino acid residue.

In some embodiments, the peptide or peptide mimetic is an amphipathic peptide. In some embodiments, the peptide or peptide mimetic has a cholesterol mediating activity and/or an ABCA1 stabilization activity and/or ABCG1 modulation activity and/or ABCG8 modulation activity and/or SR-B1 modulation activity.

The terms “polypeptide”, “peptide”, “amino acid sequence” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

In some embodiments, the peptide or a peptide mimetic is capable of binding a lipoprotein. In some embodiments, the peptide or a peptide mimetic is capable of binding a phospholipid surface selected from the group consisting of chylomicrons, HDL, LDL, and VLDL.

In some embodiments, the peptide or a peptide mimetic of the present invention exhibit a specific arrangement of amino acid residues which results in at least one amphipathic helix. The specific positioning of negatively-charged, positively-charged, and hydrophobic residues determines the formation of the amphipathic helix, and thus the intended functioning of the peptide or a peptide mimetic. In some embodiments, the peptide or a peptide mimetic comprising at least one amphipathic helix is an apolipoprotein, an HDL-binding fragment or functional fragment thereof. In some embodiments, the peptide or a peptide mimetic comprising at least one amphipathic helix is an apolipoprotein mimetic. The term “apolipoprotein” or Apo” refers to any one of several helical proteins that can combine with a lipid (i.e., solubilize the lipid) to form a lipoprotein and are a constituent of chylomicrons, HDL, LDL, and VLDL. Apolipoproteins exert their physiological effect on lipid metabolism by binding to and activating specific enzymes or transporting proteins or lipids on the cell membranes (e.g., via the ABC transporters). Apolipoproteins include, e.g., Apo A-1, Apo A-II, Apo A-IV, Apo C-1, Apo C-II, Apo C-III, Apo E, and serum amyloid proteins such as, serum amyloid A. Examples of apolipoproteins and apolipoproteins mimetic that can be used in the present invention and methods of their preparation are described in Sparrow, et al. (Peptides, Eds, Rich & Gross, p. 253-256, 1981); Kaiser and Kezdy (PNAS, USA 80:1137-1143, 1983; Science 223:249-251, 1984); Kanellis et al. (Jour Biol. Chem. 255:11464-11472, 1980); Segrest et al. (Jour Biol. Chem. 258:2290-2295, 1983); Mishra et al. (Biochemistry 37:10313-24, 1998; Jour Biol. Chem. 283:34393-34402, 2008); Gillote et al. (Jour Biol. Chem. 274:2021-28, 1999); Epand et al. (Jour Biol. Chem. 262(19): 9389-9396); Chung et al. (Jour Biol. Chem. 260(18): 10226-62); WO 2005/058938, EP 07874411.7; U.S. Pat. No. 4,643,988 U.S. Pat. No. 5,733,879, U.S. Pat. No. 6,004,925, the content of which is incorporated by reference in its entirety.

In one embodiment, the peptides or a peptide mimetic of the present invention have a cholesterol efflux mediating activity and/or an ABC transporter modulation/stabilization activity (e.g., an ABCA1 stabilization activity or an ABCA7 stabilization activity). The peptides comprise at least an amphipathic alpha helix from an apolipoprotein. In some embodiments, the peptides comprise at least an amphipathic alpha helix from a protein selected from: Apo A-I, Apo A-11, Apo A-IV, Apo E, Apo C-1, Apo C-II, Apo C-III, serum amyloid A, and combinations thereof. In some embodiments, the peptides comprise at least an amphipathic alpha helix selected from the group consisting of the first and last helices of the intact Apo A-I, and the C terminal domain of Apo E. In some embodiments, the helix comprises at least 18 amino acids, a polar face, and a nonpolar face. The polar face comprises an alignment of at least 3 acidic amino acids positioned at every 2-3 helical turns. In some embodiments, the peptide comprises at least one amino acid substitution, insertion, or deletion in the native Apo A-1, Apo A-II, Apo A-IV, Apo E, Apo C-1, Apo C-II, Apo C-III, or serum amyloid A sequence to create the alignment of acidic amino acids. In some embodiments, at least one native amino acid residue at or near the polar/nonpolar interface of the amphipathic alpha helix is substituted with a cysteine. In some embodiments, the peptides comprise at least 1, 2, 3, 4, 5, 6, 7, 8, 9, 10, or more D amino acids. In some embodiments, the carboxy terminus and the amino terminus of the peptide each comprise a D amino acid. In some embodiments, the peptides comprise all D amino acids. In some embodiments, helix comprises a sequence selected from: helix 1 (amino acids 44-65) of Apo A-I, helix 6 (amino acids 145-162) of Apo A-1, helix 7 (amino acids 167-184) of Apo A-I, helix 9 (amino acids 209-219) of Apo A-I, helix 10 (amino acids 220-238) of Apo A-I, amino acids 1-51 of Apo A-11, amino acids 5-32 of Apo A-II, amino acids 62-94 of Apo A-IV, amino acids 66-90 of Apo A-IV, amino acids 183-204 of Apo A-IV, amino acids 183-226 of Apo A-IV, amino acids 205-226 of Apo A IV, amino acids 161-204 of Apo A-IV, amino acids 161-182 of Apo A-IV, amino acids 205-248 of Apo A-IV, amino acids 227-248 of Apo A-IV, amino acids 117-138 of Apo A-IV, amino acids 138-160 of Apo A-IV, amino acids of 25-57 Apo C-1, amino acids 6-27 of Apo C-I, amino acids 29-53 of Apo C-I, amino acids 12-42 of Apo C-II, amino acids 16-40 of Apo C-II, amino acids 43-68 of Apo C-II, amino acids 37-69 of Apo C-III, amino acids 45-69 of Apo C-III, the C terminal domain (amino acids 216-299) of Apo E, amino acids 216-248 of Apo E, amino acids 216-237 of Apo E, amino acids 238-266 of Apo E, a amino acids 267-299 of Apo E, amino acids 238-263 of Apo E, amino acids 1-36 of serum amyloid A, amino acids 1-34 of serum amyloid A amino acids 5-29 of serum amyloid A, and amino acids 53-78 of serum amyloid A.

In some embodiments, the peptide comprise a sequence selected from:

(SEQ ID NO: 1)
PALEDLRQGLLPVLESFCVKFLSALEEYTKKLN;
(SEQ ID NO: 2)
PVLESFKVSFLSALEEYKTKLESALN;
(SEQ ID NO: 3)
QQARGWVTDGFSSLKDYWSTVKDKFSEFWDLDP;
(SEQ ID NO: 4)
ARMEEMGSRTRDRLDEVKEQVAEVRAKLEEQAQQIRLQAEAFQARLKSW
FEPLVE;
(SEQ ID NO: 5)
DMQRQWAGLV EKVQAAVGTS AAPVPSDNH;
(SEQ ID NO: 6)
ARMEEMGSRTRDRLDEVKEQVAEVRAKLEEQAQ;
(SEQ ID NO: 7)
ARMEEMGSRTRDRLDEVKEQVA;
(SEQ ID NO: 8)
EVRAKLEEQAQQIRLQAEAFQARLKSWFEPVLE;
(SEQ ID NO : 9)
PLVEDMQRQWAGLVEKVQAAVGTSAAPVPSDNH;
(SEQ ID NO: 10)
EVRAKLEEWFQQIRLQAEEFQARLKS;
(SEQ ID NO: 11)
PFATELHERLAKDSEKLKEEIGKELEELRARLL;
(SEQ ID NO: 12)
ELHERLAKDSEKLKEEIGKELEELR;
(SEQ ID NO: 13)
PHADELKAKIDQNVEELKGRLTPYADEFKVKIDQTVEELRRSLA;
(SEQ ID NO: 14)
PHADELKAKID QNVEELKGRLT;
(SEQ ID NO: 15)
PYADEFKVKID QTVEELRRSLA;
(SEQ ID NO: 16)
PYADEFKVKIDQTVEELRRSLA PYAQDTQEKLNHQLEGLTFQMK;
(SEQ ID NO: 17)
PYAQDTQEKLNHQLEGLTFQMK;
(SEQ ID NO: 18)
PYAQDTQEKLNHQLEGLTFQMK KNAEELKARISASAEELRQRLA;
(SEQ ID NO: 19)
KNAEELKARISASAEELRQRLA;
(SEQ ID NO: 20)
PYADQLRTQVN TQAEQLRRQLT;
(SEQ ID NO: 21)
PLAQRMERVLR ENADSLQASLR;
(SEQ ID NO: 22)
LISRIKQSELSAKMREWFSETFQKVKEKLKIDS;
(SEQ ID NO: 23)
SALDKLKEFGNTLEDKARELIS;
(SEQ ID NO: 24)
IKQSELSAKMREWFSETFQKVKEKL
(SEQ ID NO: 25)
PTFLTQVKESLSSYWESAKTAAQNLYEKTYL;
(SEQ ID NO: 26)
TQVKESLSSYWESAKTAAQNLYEKT;
(SEQ ID NO: 27)
PAVDEKLRDLYSKSTAAMSTYTGIFT;
(SEQ ID NO: 28)
QQARGWVTDGFSSLKDYWSTVKDKFSEFWDLDP;
(SEQ ID NO: 29)
DGFSSLKDYWSTVKDKFSEFWDLDP;
(SEQ ID NO: 30)
QAKEPCVESLVSQYFQTVTDYGKDLMEKVKSPELQAEAKSYFEKSKEQL
TP;
(SEQ ID NO: 31)
PCVESLVSQYFQTVTDYGKDLMEKVKSP;
(SEQ ID NO: 32)
RSFFSFLGEAFDGARDMWRAYSDMREANYI GSDKYF;
(SEQ ID NO: 33)
RSFFSFLGEAFDGARDMWRAYSDMREANYIGSDK;
(SEQ ID NO: 34)
SFLGEAEFDGARDMWRAYSDMREANY;
(SEQ ID NO: 35)
WAAEVISNARENIQRLTGHGAEDSLA;
(SEQ ID NO: 36)
PALEDLRQGLLPVLESFKVSFLSALEEYTKKLN;
(SEQ ID NO:)
LKLLDNWDSVTSTFSKLREQLGPVTQEFWDNLEKETEGLRQEMS;
(SEQ ID NO: 37)
LKLLDNWDSVTSTFSKLREQLGPALEDLRQGLL;
(SEQ ID NO: 38)
ARLAEYHAKATEHLSTLSEKAKPVLESFKVSFLSALEEYTKKLN;
(SEQ ID NO: 39)
PYSDELRQRLAARLEALKENGGPVLESFKVSFLSALEEYTKKLN;
(SEQ ID NO: 40)
PLGEEMRDRARAHVDALRTHLAPVLESFKVSFLSALEEYTKKLN;

and

(SEQ ID NO: 41)
PALEDLRQGLLLKLLDNWDSVTSTFSKLREQLG.

In some embodiments, the peptide comprise a sentence selected from:

| | |
|---|---|
| DWFKAFYDKVAEKFKEAF; | (SEQ ID NO: 42) |
| DWLKAFYDKVAEKLKEAF. | (SEQ ID NO: 43) |

In some embodiments, the peptide comprise a sequence selected from:

| | |
|---|---|
| EVRSKLEEWFAAFREFAEEFLARLKS; | (SEQ ID NO: 44) |
| EVRSKLEEWFAAFREFFEEFLARLKS; | (SEQ ID NO: 45) |
| EFRSKLEEWFAAFREFFEEFLARLKS; | (SEQ ID NO: 45) |
| EFRSKLEEWFAAFREFAEEFLARLKS. | (SEO ID NO: 46) |

In some embodiments, the peptides or peptide mimetic further comprise a second amphipathic alpha helix as described herein. In some embodiments, the first and the second amphipathic helices comprise a sequence selected from the group consisting of: helix 1 (amino acids 44-65) of Apo A-1 and helix 9 (amino acids 209-219) of Apo A-I linked in order; helix 9 (amino acids 209-219) of Apo A-1 and helix 1 (amino acids 44-65) of Apo A-1 linked in order; helix 6 (amino acids 145-162) of Apo A-I and helix 10 (amino acids 220-238) of Apo A-I linked in order; helix 7 (amino acids 167-184) of Apo A-I and helix 10 (amino acids 220-238) of Apo A-I linked in order; helix 9 (amino acids 201-219) of Apo A-1 and helix 10 (amino acids 220-238) of Apo A-I linked in order; helix 6 (amino acids 145-162) of Apo A-1 and helix 7 (amino acids 167-184) of Apo A-I linked in order; helix 1 (amino acids 44-65) of Apo A-I and helix 2 (amino acids 66-87) of Apo A-1 linked in order; helix 8 (amino acids 185-209) of Apo A-1 and helix 10 (amino acids 220-238) of Apo A-I linked in order; and the C terminal domain of Apo E (amino acids 216-299). In some embodiments, the peptides or peptide mimetic comprise a first and second amphipathic alpha helix independently selected from the group consisting of the first and last helices of the intact Apo A-I, and the C terminal domain of Apo E. In some embodiments, the peptides or peptide mimetic comprise tandem amphipathic helices from Apo A-I. In some embodiments, the peptides or peptide mimetic comprise tandem amphipathic helices from Apo A-I selected from the group consisting of helices 1-2; and helices 9-10.

Samples and Sampling

The methods involve analysis of one or more samples from an individual. An individual or a patient is any multi-cellular organism; in some embodiments, the individual is an animal, e.g., a mammal. In some embodiments, the individual is a human.

The sample may be any suitable type that allows for the analysis of RCT pathway. Samples may be obtained once or multiple times from an individual. Multiple samples may be obtained from different locations in the individual (e.g., blood samples, bone marrow samples and/or atherosclerotic plaque samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement treatment response and also the monitoring for disease.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cardiovascular disease may be sampled (e.g., by blood draw) relatively frequently (e.g., every month or every three months) for the first six months to a year after treatment, then, as treatment improved the condition, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

In one embodiment, a sample may be obtained from an apparently healthy individual during a routine checkup and analyzed so as to provide an assessment of the individual's general health status. In another embodiment, a sample may be taken to screen for commonly occurring diseases. Such screening may encompass testing for a single disease, a family of related diseases or a general screening for multiple, unrelated diseases. Screening can be performed weekly, bi-weekly, monthly, bi-monthly, every several months, annually, or in several year intervals and may replace or complement existing screening modalities.

In another embodiment, an individual with a known increased probability of disease occurrence may be monitored regularly to detect for the appearance of a particular disease or class of diseases. An increased probability of disease occurrence can be based on familial association, age, previous genetic testing results, or occupational, environmental or therapeutic exposure to disease causing agents. For example is the presence of inherited mutations that predispose individuals to a particular condition. Individuals with increased risk for specific diseases can be monitored regularly for the first signs of a condition. Monitoring can be performed weekly, bi-weekly, monthly, bi-monthly, every several months, annually, or in several year intervals, or any combination thereof. Monitoring may replace or complement existing screening modalities. Through routine monitoring, early detection of the presence of disease may result in increased treatment options including treatments with lower toxicity and increased chance of disease control or cure.

In these embodiments, one or more samples may be taken from the individual, and subjected to a modulator, as described herein. In some embodiments, the sample is divided into subsamples that are each subjected to a different modulator. After treatment with the modulator, different discrete cell populations in the sample or subsample are analyzed to determine function of RCT pathways. Any suitable form of analysis that allows a determination of RCT pathways may be used. In some embodiments, the analysis includes the determination of lipid efflux, e.g. cholesterol and phospholipids. In some embodiments, the analysis includes the determination of phospholipids.

Certain fluid samples can be analyzed in their native state with or without the addition of a diluent or buffer. Alternatively, fluid samples may be further processed to obtain enriched or purified discrete cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 μm, as disclosed in U.S. patent application Ser. No. 09/790,673. Alternatively, whole blood can be separated into its constituent cells based on size, shape, deformability or surface receptors or surface antigens by the use of a microfluidic device as disclosed in U.S. patent application Ser. No. 10/529,453.

Select cell populations can also be enriched for or isolated from whole blood through positive or negative selection based on the binding of antibodies or other entities that recognize cell surface or cytoplasmic constituents.

Solid tissue samples may require the disruption of the extracellular matrix or tissue stroma and the release of single cells for analysis. Various techniques are known in the art including enzymatic and mechanical degradation employed separately or in combination. An example of enzymatic dissociation using collagenase and protease can be found in Wolters G H J et al. An analysis of the role of collagenase and protease in the enzymatic dissociation of the rat pancrease for islet isolation. Diabetologia 35:735-742, 1992. Examples of mechanical dissociation can be found in Singh, NP. Technical Note: A rapid method for the preparation of single-cell suspensions from solid tissues. Cytometry 31:229-232 (1998). Alternately, single cells may be removed from solid tissue through microdissection including laser capture microdissection as disclosed in Laser Capture Microdissection, Emmert-Buck, M. R. et al. Science, 274(8):998-1001, 1996.

The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 61/048,886; 61/048,920; and 61/048,657. See also, the commercial products from companies such as BD and BCI as identified above.

In some embodiments, the cells are cultured post collection in a media suitable for measuring RCT function (e.g. RPMI, DMEM) in the presence, or absence, of serum such as fetal bovine serum, bovine serum, human serum, porcine serum, horse serum, or goat se

Macrophages and Foam Cells

In some embodiments, the present invention provides methods and compositions for prognosing, diagnosing, and/or predicting a response to treatment of a condition associated with a deficiency in a reverse cholesterol transport (RCT) pathway by contacting a macrophage or macrophage like cell with a modulator that is specific for a RCT pathway. In some embodiments, the invention provides methods and compositions for the screening of compounds for treatment of a condition associated with RCT deficiency and/or assessing risk of toxicity of a treatment of a condition associated with RCT deficiency by contacting a macrophage or macrophage like cell with a modulator that is specific for a RCT pathway. In some embodiments, the invention provides methods and compositions to identify new druggable targets for the treatment of a condition associated with RCT deficiency by contacting a macrophage or macrophage like cell with a modulator that is specific for a RCT pathway. In some embodiments, the present invention a method of treating a RCT related disease comprising administering to a subject in need thereof an effective amount of a modulator that is specific for a RCT pathway.

Macrophages are released from the bone marrow as immature monocytes, and after circulating in the blood stream, migrate into tissues to undergo final differentiation into resident macrophages, including Kupffer cells in the liver, alveolar macrophages in the lung, and osteoclasts in the bone. Monocytes and macrophages are phagocytes, acting in innate immunity as well as to help adaptive immunity of vertebrate animals. Their role is to phagocytose (engulf and then digest) cellular debris and pathogens either as stationary or mobile cells, and to stimulate lymphocytes and other immune cells to respond to the pathogen. They can be identified by specific expression of a number of proteins including CD14, CD11b, F4/80 (mice)/EMR1 (human), Lysozyme M, MAC-1/MAC-3 and CD68 by flow cytometry or immunohistochemical staining (Khazen W, et al. 2005 FEBS Lett. 579 (25): 5631-4). When a monocyte enters damaged tissue through the endothelium of a blood vessel (a process known as the leukocyte extravasation), it undergoes a series of changes to become a macrophage. Monocytes are attracted to a damaged site by chemical substances through chemotaxis, triggered by a range of stimuli including damaged cells, pathogens and cytokines released by macrophages already at the site. At some sites such as the testis, macrophages have been shown to populate the organ through proliferation. Unlike short-lived neutrophils, macrophages survive longer in the body up to a maximum of several months.

Macrophages perform a multitude of functions essential for tissue remodeling, inflammation, and immunity, including but not limited to phagocytosis, cytotoxicity, and secretion of a variety of cytokines, growth factors, lysozymes, proteases, complement components, coagulation factors, and prostaglandins.

Monocyte is a type of white blood cell. Monocytes have two main functions in the immune system: (1) replenish resident macrophages and dendritic cells under normal states, and (2) in response to inflammation signals, monocytes can move quickly to sites of infection in the tissues and divide/differentiate into macrophages and dendritic cells to elicit an immune response. Monocytes are produced by the bone marrow from haematopoietic stem cell precursors called monoblasts. Monocytes circulate in the bloodstream for about one to three days and then typically move into tissues throughout the body. In the tissues monocytes mature into different types of macrophages at different anatomical locations. Monocytes which migrate from the bloodstream to other tissues will then differentiate into tissue resident macrophages or dendritic cells. Macrophages are responsible for protecting tissues from foreign substances but are also suspected to be the predominant cells involved in triggering atherosclerosis. They are cells that possess a large smooth nucleus, a large area of cytoplasm and many internal vesicles for processing foreign material.

Foam cells are cells in an atheroma derived from both macrophages and smooth muscle cells which have accumulated low density lipoproteins, LDLs, by endocytosis. The LDL has crossed the endothelial barrier and has been oxidized by reactive oxygen species produced by the endothelial cells. Foam cells can also be known as fatty like streaks and typically line the tunica intima of the vasculature.

Foam cells are not dangerous as such, but can become a problem when they accumulate at particular foci thus creating a necrotic centre of the atherosclerosis. If the fibrous cap that prevents the necrotic centre from spilling into the lumen of a vessel ruptures, a thrombus can form which can lead to emboli occluding smaller vessels. The occlusion of small vessels results in ischemia, and contributes to stroke and myocardial infarction, two of the leading causes of cardiovascular-related death.

Foam cells (macrophages laden with lipid) are culpable in early and late stage development of atherosclerotic lesions in arterial walls. Foam cells arise from an imbalance in lipid uptake and efflux. Multiple mechanisms (e.g. free diffusion, membrane bound ABCA1 transporter activity, and lipoprotein receptors) regulate lipid accumulation. Thus, activation of ABCA1 to prevent cholesterol and lipid deposition and accumulation in macrophages and arterial walls is a worthwhile goal in the treatment of atherogenesis.

RCT-Related Conditions

In one aspect, the present invention provides methods and compositions for diagnosis, prognosis, prediction of outcome of a treatment, and/or drug screening of a condition associated with a RCT deficiency.

A. Cardiovascular Diseases

In some embodiments, the present invention provides methods of diagnosis, prognosis, prediction of outcome of a treatment, identification of druggable targets and/or drug screening of a cardiovascular disease.

Cardiovascular disease refers to the class of diseases that involve the heart or blood vessels (arteries and veins). While the term technically refers to any disease that affects the cardiovascular system, it is usually used to refer to those related to atherosclerosis (arterial disease). These conditions have similar causes, mechanisms, and treatments.

In some embodiments, the present invention provides methods of diagnosis, prognosis, prediction of outcome of a treatment, and/or drug screening of atherosclerosis.

Atherosclerosis is the condition in which an artery wall thickens as the result of a build-up of fatty materials such as cholesterol. It is a syndrome affecting arterial blood vessels, a chronic inflammatory response in the walls of arteries, in large part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins (HDL). It is caused by the formation of multiple plaques within the arteries (Maton, et al. (1993). Human Biology and Health. Englewood Cliffs, N.J., USA: Prentice Hall). The atheromatous plaque is divided into three distinct components: the atheroma, which is the nodular accumulation at the center of large plaques, composed of macrophages nearest the lumen of the artery; underlying areas of cholesterol crystals; and calcification at the outer base of older/more advanced lesions.

The first step of atherogenesis is the development of fatty streaks, which are small sub-endothelial deposits of monocyte-derived macrophages. The primary documented driver of this process is oxidized lipoprotein particles within the wall, beneath the endothelial cells, though upper normal or elevated concentrations of blood glucose also plays a major role and not all factors are fully understood. Fatty streaks may appear and disappear. Low Density Lipoprotein particles in blood plasma, when they invade the endothelium and become oxidized create a risk for cardiovascular disease. A complex set of biochemical reactions regulates the oxidation of LDL, chiefly stimulated by presence of enzymes, e.g. Lp-LpA2 and free radicals in the endothelium or blood vessel lining.

The initial damage to the blood vessel wall results in an inflammatory response. Monocytes enter the artery wall from the bloodstream, with platelets adhering to the area of insult. This may be promoted by redox signaling induction of factors such as VCAM-1, which recruit circulating monocytes. The monocytes differentiate macrophages, which ingest oxidized LDL, slowly turning into large “foam cells”—so-described because of their changed appearance resulting from the numerous internal cytoplasmic vesicles and resulting high lipid content. Foam cells eventually die, and further propagate the inflammatory process. There is also smooth muscle proliferation and migration from tunica media to intima responding to cytokines secreted by damaged endothelial cells. This would cause the formation of a fibrous capsule covering the fatty streak.

In terms of treatment for atherosclerosis, in general, the group of medications referred to as statins has been the most popular and are widely prescribed for treating atherosclerosis. The statins, and some other medications, have been shown to have antioxidant effects, possibly part of their basis for some of their therapeutic success in reducing cardiac events. Combinations of statins, niacin, intestinal cholesterol absorption-inhibiting supplements (ezetimibe and others, and to a much lesser extent fibrates) have been the most successful in changing common but sub-optimal lipoprotein patterns and group outcomes. Diet and dietary supplements are also used to help treat atherosclerosis. For example, vitamin C acts as an antioxidant in vessels and inhibits inflammatory process (Böhm F, et al. (2007) Atherosclerosis 190 (2): 408-15). Patients at risk for atherosclerosis-related diseases are increasingly being treated prophylactically with low-dose aspirin and a statin.

The actions of macrophages drive atherosclerotic plaque progression Immunomodulation of atherosclerosis is the term for techniques which modulate immune system function in order to suppress this macrophage action (Jan Nilsson; et al (2005) Arteriosclerosis, Thrombosis, and Vascular Biology 5: 18-28). In some embodiments, the present invention provides a method of treating atherosclerosis by modulating macrophage accumulation or activation with an oxidative agent, such as chlorite. In some embodiments, the macrophage activation is reduced or inhibited.

In general, the group of medications referred to as statins has been the most popular and are widely prescribed for treating atherosclerosis. Combinations of statins, niacin, intestinal cholesterol absorption-inhibiting supplements (ezetimibe and others, and to a much lesser extent fibrates) have been the most successful in changing common but sub-optimal lipoprotein patterns and group outcomes. Patients at risk for atherosclerosis-related diseases are increasingly being treated prophylactically with low-dose aspirin and a statin.

In some embodiments, the present invention provides a method of treating hypertension comprising administering to a subject in need thereof an effective amount of a modulator that is specific for a reverse cholesterol transporter.

Hypertension, also referred to as high blood pressure, is a medical condition in which the blood pressure is chronically elevated. It normally refers to arterial hypertension. Hypertension is related to hyperglycemia and hyperlipidemia. In normotensive individuals, insulin may stimulate sympathetic activity without elevating mean arterial pressure. However, in more extreme conditions such as that of the metabolic syndrome, the increased sympathetic neural activity may over-ride the vasodilatory effects of insulin. Insulin resistance and/or hyperinsulinemia have been suggested as being responsible for the increased arterial pressure in some patients with hypertension.

There are many classes of medications for treating hypertension, together called antihypertensives, which, by varying means, act by lowering blood pressure. Evidence suggests that reduction of the blood pressure by 5-6 mmHg can decrease the risk of stroke by 40%, of coronary heart disease by 15-20%, and reduces the likelihood of dementia, heart failure, and mortality from cardiovascular disease. Common drugs for treating hypertension include but are not limited to ACE inhibitors, angiotensin II receptor antagonists, alpha blockers, beta blockers calcium channel blockers, direct renin inhibitors, and diuretics.

The subject methods can be used to diagnose and treat other cardiovascular diseases including but not limited to aneurysm, angina, stroke, cerebrovascular disease, congestive heart failure, coronary artery disease, myocardial infarction (heart attack) and peripheral vascular disease.

B. Neurological Diseases

In some embodiments, the present invention provides methods of diagnosis, prognosis, prediction of outcome of a treatment, identification of a druggable target and/or drug screening of a neurological disease.

1. Alzheimer's Disease (AD)

In some embodiments, the present invention provides methods for diagnosis, prognosis, prediction of outcome of a treatment, identification of a druggable target and/or drug screening of Alzheimer's disease. Alzheimer's disease (AD), also called Alzheimer disease, Senile Dementia of the Alzheimer Type (SDAT) or simply Alzheimer's, is the most common form of dementia. Generally it is diagnosed in people over 65 years of age, although the less-prevalent early-onset Alzheimer's can occur much earlier. Although each sufferer experiences Alzheimer's in a unique way, there are many common symptoms. The earliest observable symptoms are often mistakenly thought to be ‘age-related’ concerns, or manifestations of stress. In the early stages, the most commonly recognized symptom is memory loss, such as difficulty in remembering recently learned facts. When a doctor or physician has been notified, and AD is suspected, the diagnosis is usually confirmed with behavioral assessments and cognitive tests, often followed by a brain scan if available. As the disease advances, symptoms include confusion, irritability and aggression, mood swings, language breakdown, long-term memory loss, and the general withdrawal of the sufferer as their senses decline (Waldemar G, Dubois B, Emre M, et al. (January 2007). Eur J Neurol 14 (1): e1-26). Gradually, bodily functions are lost, ultimately leading to death. Individual prognosis is difficult to assess, as the duration of the disease varies. AD develops for an indeterminate period of time before becoming fully apparent, and it can progress undiagnosed for years.

Research indicates that the disease is associated with plaques and tangles in the brain (Tiraboschi P, Hansen L A, Thal L J, Corey-Bloom J (June 2004). Neurology 62 (11): 1984-9). The disease course is divided into four stages, with a progressive pattern of cognitive and functional impairment: pre-dementia, early dementia, moderate dementia, and advanced dementia Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Both amyloid plaques and neurofibrillary tangles are clearly visible by microscopy in brains of those afflicted by AD.

Alzheimer's disease has been identified as a protein misfolding disease (proteopathy), caused by accumulation of abnormally folded A-beta and tau proteins in the brain (Hashimoto M, Rockenstein E, Crews L, Masliah E (2003) Neuromolecular Med. 4 (1-2): 21-36). Plaques are made up of small peptides, 39-43 amino acids in length, called beta-amyloid (also written as A-beta or Aβ). Beta-amyloid is a fragment from a larger protein called amyloid precursor protein (APP), a transmembrane protein that penetrates through the neuron's membrane. APP is critical to neuron growth, survival and post-injury repair (Priller C, et al. 2006 J. Neurosci. 26 (27): 7212-21). In Alzheimer's disease, an unknown process causes APP to be divided into smaller fragments by enzymes through proteolysis (Hooper N M (April 2005) Biochem. Soc. Trans. 33 (Pt 2): 335-8). One of these fragments gives rise to fibrils of beta-amyloid, which form clumps that deposit outside neurons in dense formations known as senile plaques (Ohnishi S, Takano K (March 2004) Cell. Mol. Life Sci. 61 (5): 511-24). AD is also considered a tauopathy due to abnormal aggregation of the tau protein. Every neuron has a cytoskeleton, an internal support structure partly made up of structures called microtubules. Tau protein stabilizes the microtubules when phosphorylated, and is therefore called a microtubule-associated protein. In AD, tau undergoes chemical changes, becoming hyperphosphorylated; it then begins to pair with other threads, creating neurofibrillary tangles and disintegrating the neuron's transport system (Hernandez F, Avila J September 2007 Cell. Mol. Life Sci. 64 (17): 2219-33).

It is not known exactly how disturbances of production and aggregation of the beta amyloid peptide gives rise to the pathology of AD. The amyloid hypothesis traditionally points to the accumulation of beta amyloid peptides as the central event triggering neuron degeneration. Accumulation of aggregated amyloid fibrils, which are believed to be the toxic form of the protein responsible for disrupting the cell's calcium ion homeostasis, induces apoptosis (Yankner B A, Duffy L K, Kirschner D A (October 1990) Science (journal) 250 (4978): 279-82). It is also known that Aβ selectively builds up in the mitochondria in the cells of Alzheimer's-affected brains, and it also inhibits certain enzyme functions and the utilization of glucose by neurons (Chen X, Yan S D (December 2006) IUBMB Life 58 (12): 686-94). Various inflammatory processes and cytokines may also have a role in the pathology of Alzheimer's disease. Inflammation is a general marker of tissue damage in any disease, and may be either secondary to tissue damage in AD or a marker of an immunological response (Greig N H, Mattson M P, Perry T, et al. (December 2004) Ann. N.Y. Acad. Sci. 1035: 290-315).

Four medications are currently approved by regulatory agencies such as the U.S. Food and Drug Administration (FDA) and the European Medicines Agency (EMEA) to treat the cognitive manifestations of AD: three are acetylcholinesterase inhibitors and the other is memantine, an N-methyl-D-aspartic acid (NMDA) receptor antagonist. Reduction in the activity of the cholinergic neurons is a well-known feature of Alzheimer's disease (Geula C, Mesulam M M (1995). Alzheimer Dis Assoc Disord 9 Suppl 2: 23-28). Acetylcholinesterase inhibitors are employed to reduce the rate at which acetylcholine (ACh) is broken down, thereby increasing the concentration of ACh in the brain and combating the loss of ACh caused by the death of cholinergic neurons (Stahl S M (2000). J Clin Psychiatry 61 (11): 813-814). Examples of the cholinesterase inhibitors approved for the management of AD symptoms include donepezil, galantamine, and rivastigmine. There is evidence for the efficacy of these medications in mild to moderate Alzheimer's disease, and some evidence for their use in the advanced stage (Birks J, Harvey R J (2006). Cochrane Database Syst Rev (1): CD001190). Only donepezil is approved for treatment of advanced AD dementia. The common side effects associated with cholinesterase inhibitors include nausea and vomiting, muscle cramps, decreased heart rate (bradycardia), decreased appetite and weight, and increased gastric acid production.

The subject methods can be used diagnosis, prognosis, prediction of outcome of a treatment, identification of druggable targets and/or drug screening of other neurological diseases including but not limited to amyotrophic lateral sclerosis, Parkinson's disease, aging, Niemann-Pick disease and Gaucher's disease.

Conditions other than the one described herein involving altered RCT pathways are also encompassed by the present invention. For example, recent studies have shown that pancreas beta-cells express ABCA1. Additionally, other studies have shown that Apo A-I facilitates increased insulin secretion from pancreas beta-cells and that this increase depended on ABCA1 expression.

Methods

In some embodiments, the invention provides methods of prognosing, diagnosing, identifying a druggable target and/or predicting a response to treatment of a condition associated with a deficiency in a reverse cholesterol transport (RCT) pathway. For example, the methods of the invention can be used to identify RCT deficiency, identify responders to receptor activation, identify responders to a certain treatment, assess treatment progress and/or to predict treatment outcome.

The methods and compositions, and kits described herein are for any condition for which a correlation between the condition, its prognosis, course of treatment, or other relevant characteristic, and the state of a RCT pathway, in samples from individuals may be ascertained.

In some embodiments, this invention is directed to methods and compositions, and kits for analysis, drug screening, diagnosis, prognosis, for methods of disease treatment and prediction of outcome. In some embodiments, the present invention involves methods of analyzing experimental data. In some embodiments, the RCT pathway state data of one or more cells in a sample (e.g. clinical sample) is used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. Hence, therapeutic regimens can be individualized and tailored according to the data obtained prior to, and at different times over the course of treatment, thereby providing a regimen that is individually appropriate. In some embodiments, a compound is contacted with cells to analyze the response to the compound. The RCT pathway state data of one or more cells can be generated by assessing lipid efflux profile, mRNA expression, protein expression, protein activation level and/or a phenotype in response to at least one RCT pathway modulator in the at least one cell. In some embodiments, the cell is a macrophage or a macrophage-like cell.

The invention allows for the determination of the RCT pathway state. The methods of the invention provide tools useful in the treatment of an individual afflicted with a condition, including but not limited to: methods for assigning a risk group, methods of predicting an increased risk of developing secondary complications, methods of choosing a therapy for an individual, methods of predicting duration of response, response to a therapy for an individual, methods of determining the efficacy of a therapy in an individual, and methods of determining the prognosis for an individual. The state of a RCT pathway can serve as a prognostic indicator to predict the course of a condition, e.g. whether the course of a condition in an individual will develop in a cardiovascular disease, thereby aiding the clinician in managing the patient and evaluating the modality of treatment to be used. In another embodiment, the present invention provides information to a physician to aid in the clinical management of a patient so that the information may be translated into action, including treatment, prognosis or prediction.

In some embodiments, the state of a RCT pathway can be used to confirm or refute the presence of a suspected genetic or physiologic abnormality associated with increased risk of disease. Such testing methodologies can replace or use in combination with other confirmatory techniques like cytogenetic analysis or fluorescent in situ histochemistry (FISH). In still another embodiment, the state of a RCT pathway can be used to confirm or refute a diagnosis of a pre-pathological or pathological condition.

In some embodiments, the methods described herein are used to screen candidate compounds useful in the treatment of a condition or to identify new drug targets.

In instances where an individual has a known pre-pathologic or pathologic condition, the status of the state of a RTC pathway can be used to predict the response of the individual to available treatment options. In one embodiment, an individual treated with the intent to reduce in number or ablate cells that are causative or associated with a pre-pathological or pathological condition can be monitored to assess the decrease in such cells and the state of a RTC pathway over time. A reduction in causative or associated cells may or may not be associated with the disappearance or lessening of disease symptoms, e.g. depending of the state of the RTC pathway. If the anticipated decrease in cell number and/or improvement in the state of a RTC pathway do not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

The invention provides methods for determining a RCT pathway state by assessing lipid efflux profile, mRNA expression, protein expression, protein activation level and/or a phenotype in response to at least one RCT pathway modulator in the at least one cell. In some embodiments, the cell is a macrophage or a macrophage-like cell. In some embodiments, the invention provides methods of prognosing, diagnosing, and/or predicting a response to treatment of a condition associated with a deficiency in a reverse cholesterol transport (RCT) pathway in a subject comprising the steps of: (a) providing a population of cells from a subject; (b) contacting the population of cells with a modulator that specifically modulates a reverse cholesterol transporter pathway; (c) assessing lipid efflux profile, mRNA expression, protein expression, protein activation level and/or a phenotype in the at least one cell treated with the modulator or a medium comprising said cell; (d) determining whether there is a deficiency in the reverse cholesterol transport pathway of the subject, where the determining is based in the assessing of lipid efflux profile, mRNA expression, protein expression, protein activation level and/or the phenotype in the at least one cell; and (e) prognosing, diagnosing, and/or predicting a response to treatment of the condition associated with a deficiency in a reverse cholesterol transport (RCT) pathway, where the prognosing, diagnosing, and/or predicting a response to treatment is based in the determining in step (d). In some embodiments, the cell is a macrophage or a macrophage-like cell. The medium comprising the cell can be, for example, tissue, organ, blood, serum, plasma, body fluid, or culture media. In some embodiments, the reverse cholesterol transport modulator is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

In some embodiments, assessing lipid efflux profile includes measuring total Cholesterol, cholesterol ester, HDL, LDL, IDL, VLDL, triglycerides ratio and phospholipids selected from the group consisting of sphingolipids and phosphatidyl choline. In some embodiments, the sphingolipids are selected from the group consisting of spingosines, ceramides and sphoingomyelings. Thus, in some embodiments, assessing lipid efflux profile includes measuring cholesterol ester, spingosines, ceramides, sphoingomyelings and phosphatidyl choline. In some embodiments, assessing lipid efflux profile includes measuring the conversion of α-mobility HDL particles to pre-β1-HDL. Thus in some embodiments, assessing lipid efflux profile includes measuring cholesterol ester, spingosines, ceramides, sphoingomyelings, phosphatidyl choline, and measuring the conversion of α-mobility HDL particles to pre-β1-HDL.

In some embodiments, the lipid efflux profile is used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. In some embodiments, the methods of the invention may further comprise comparing the content of one or more lipids in the lipid efflux profile to a predetermined threshold value. In some embodiments, when the lipid efflux profile is above or below a predetermined threshold is an indication that can be used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. For example, a decrease of at least 20% or more of cholesterol ester and/or shingolipids content in tissue can be used as an indication of a good prognosis, diagnosis and/or treatment outcome. In some embodiments, the threshold is at least 20%, 25%, 30%, 40%, 50%, 60%, 70%, 80% or 90% reduction of a lipid content in tissue when compared to a control sample. In some embodiments, the threshold is at least 20%, 25%, 30%, 40%, 50%, 60%, 70%, 80% or 90% increase of a lipid content in tissue when compared to a control sample. In some embodiments, the threshold is at least 20%, 25%, 30%, 40%, 50%, 60%, 70%, 80% or 90% reduction of a lipid content in plasma or serum when compared to a control sample. In some embodiments, the threshold is at least 20%, 25%, 30%, 40%, 50%, 60%, 70%, 80% or 90% increase of a lipid content in plasma or serum when compared to a control sample. In some embodiments, the threshold is at least 20%, 25%, 30%, 40%, 50%, 60%, 70%, 80% or 90% reduction of a lipid content in a cell when compared to a control sample. In some embodiments, the threshold is at least 20%, 25%, 30%, 40%, 50%, 60%, 70%, 80% or 90% increase of a lipid content in a cell when compared to a control sample. In some embodiments, the lipid is cholesterol. In some embodiments, the lipid is a phospholipid. In some embodiments, the phospholipid is a sphingolipids. In some embodiments, the sphingolipids are selected from the group consisting of spingosines, ceramides and sphoingomyelings. Thus, in some embodiments, when the content of cholesterol ester, spingosines, ceramides, sphoingomyelings and phosphatidyl choline is above or below a predetermined threshold in tissue, blood, plasma, serum and/or a cell is an indication that can be used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. In some embodiments, the lipid is the conversion of α-mobility HDL particles to pre-β1-HDL. Thus in some embodiments, when the conversion of α-mobility HDL particles to pre-β1-HDL is above or below a predetermined threshold in tissue, blood, plasma, serum and/or a cell is an indication that can be used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents.

In some embodiments, the ratio of several lipid components in the lipid efflux profile can be used is an indication that can be used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. For example, in some embodiments, the ratio of cholesterol ester to a shingolipid can be used as an indication of a good prognosis, diagnosis and/or treatment outcome. In some embodiments, the ratio can be 0.001:1 to 1:1. Without limiting the scope of the invention, the ratio of one or more lipid components can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1. In some embodiments, the lipid components are cholesterol and a phospholipid. In some embodiments the lipid components are cholesterol and the conversion of α-mobility HDL particles to pre-β1-HDL. In some embodiments, the lipid components are a phospholipid and the conversion of α-mobility HDL particles to pre-β1-HDL. In some embodiments, the phospholipid is a sphingolipids. In some embodiments, the sphingolipids are selected from the group consisting of spingosines, ceramides and sphoingomyelings.

The invention provides methods for determining a RCT pathway state by assessing mRNA expression, protein expression, and/or protein activation level in response to at least one RCT pathway modulator in the at least one cell. Examples of gene and/or proteins that can be measured in the methods described herein include, but are not limited to, kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, regulators of translation, growth factors, cytokines, immune modulators, and hormones.

In some embodiments, the invention provides methods for determining a RCT pathway state by protein expression in response to at least one RCT pathway modulator in the at least one cell. In some embodiments, the protein is an inflammatory protein. In some embodiments, the proteins are selected from the group consisting of CRP, Fibrinogen, Haptoglobin, IL-18, SAP (serum amyloid P component), Rantes, TIMP-1, VCAM-1, MIP-1beta, MPO, VEGF-alpha and IL-7.

In some embodiments, the methods of the invention may further comprise comparing the mRNA expression, protein expression, and/or protein activation level to a predetermined threshold value. In some embodiments, when the mRNA expression, protein expression, and/or protein activation level is above or below a predetermined threshold is an indication that can be used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. For example a decreased of at least 10% in plasma of CRP, Fibrinogen, Haptoglobin, IL-18, SAP (serum amyloid P component), Rantes, TIMP-1, VCAM-1, MIP-1beta, MPO, VEGF-alpha or IL-7 can be used as an indication of a good prognosis, diagnosis and/or treatment outcome. In some embodiments, the threshold is at least 5%, 10%, 20%, 25%, 30%, 40%, 50%, 60%, 70%, 80% or 90% reduction in tissue when compared to a control sample. In some embodiments, the threshold is at least 5%, 10%, 20%, 25%, 30%, 40%, 50%, 60%, 70%, 80% or 90% increase in tissue when compared to a control sample. In some embodiments, the threshold is at least 5%, 10%, 20%, 25%, 30%, 40%, 50%, 60%, 70%, 80% or 90% reduction content in plasma or serum when compared to a control sample. In some embodiments, the threshold is at least 5%, 10%, 20%, 25%, 30%, 40%, 50%, 60%, 70%, 80% or 90% increase in plasma or serum when compared to a control sample. In some embodiments, the threshold is at least 5%, 10%, 20%, 25%, 30%, 40%, 50%, 60%, 70%, 80% or 90% reduction in a cell when compared to a control sample. In some embodiments, the threshold is at least 5%, 10%, 20%, 25%, 30%, 40%, 50%, 60%, 70%, 80% or 90% increase in a cell when compared to a control sample.

In some embodiments, the invention provides methods for determining a RCT pathway state by assessing a combination of one or more lipid, one or more mRNAs, and one or more proteins. For example, a RCT pathway state can be assessed my measuring cholesterol, one or more phospholipids, and the expression of one or more proteins. Thus, in some embodiments, a RCT pathway state can be assessed by measuring a combination of readouts. In some embodiments, a RCT pathway state is assessed by measuring cholesterol, conversion of α-mobility HDL particles to pre-β1-HDL, a sphingolipid selected from the group consisting of spingosines, ceramides and sphoingomyelings, and a CRP, Fibrinogen, Haptoglobin, IL-18, SAP (serum amyloid P component), Rantes, TIMP-1, VCAM-1, MIP-1beta, MPO, VEGF-alpha or IL-7

The lipid efflux profile, mRNA expression, protein expression, protein activation level and/or a cell phenotype in response to at least one RCT pathway modulator can be assessed by any suitable method known in the art. Isolation of HDL fraction or subfraction thereof may be performed by any known method in the art, including the methods described herein. For example HDL fraction or subfraction thereof may be prepared by density ultracentrifugation, as described in Mendez, A. J (1991), from plasma or serum. Other methods are also described in Chapman M J. et al. (1981), Guerin M. et al. (2001 and 2002), Rainwater D L. et al. (1998), Cheung M C et al. (1987), Duriez P et al. (1999), Li Z et al. (1994), and Asztalos B F. et al. (1993).

For example, any biomarker may be measured by using standard immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays, immunoelectrophoresis; immunoprecipitation. Any biomarker can be measured by gas chromatography, high performance liquid chromatography (HPLC), size exclusion chromatography, solid-phase affinity, nuclear magnetic resonance (NMR) spectroscopy, laser spectrophotometry, laser spectroscopy, liquid scintillation counting, LC/MS-MS, etc.

Any biomarker may be determined by various mass spectrometric methods, including but not limited to, gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix-assisted laser desorption-time of flight-MS, Fourier-transform-ion-cyclotron-resonance-MS, cycloidal-MS, and the like. In addition, two or more mass spectrometers may be coupled (e.g., MS/MS, LC/MS-MS) first to separate precursor ions, then to separate and measure gas phase fragment ions. In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In addition, any biomarker can be measured using liquid scintillation counting, geiger counting, CCD based detection, film based detection, and others.

In some embodiments, the invention provides methods of screening of compounds for treatment of a condition associated with reverse cholesterol transport deficiency and/or assessing risk of toxicity of a treatment of a condition associated with reverse cholesterol transport deficiency comprising the steps of: (a) providing a cell; (b) contacting the macrophage or macrophage-like cell with one or more compounds, wherein said one or more compounds are possible candidates for the treatment of a condition associated with reverse cholesterol transport deficiency, and/or wherein said one or more compounds are used in the treatment of a condition associated with reverse cholesterol transport deficiency; (c) assessing lipid efflux profile, mRNA expression, protein expression, protein activation level and/or a phenotype in said cell treated with said compound or a medium comprising said cell; and (d) selecting said one or more compounds for treatment of said condition associated with reverse cholesterol transport deficiency and/or determining toxicity of a treatment of said condition associated with reverse cholesterol transport deficiency, wherein said selecting and/or said determining are based in said assessing from step (c). In some embodiments, the cell is a macrophage or a macrophage-like cell. The medium comprising the cell can be, for example, tissue, organ, blood, serum, plasma, body fluid, or culture media. In some embodiments, the reverse cholesterol transport modulator is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

The compound may be a single agent or compound. Alternatively, the compound may be a combination of agents or compounds. The compound also may be a single agent or compound or a combination of agents or compounds together with some other intervention, such as a lifestyle change (e.g., change in diet, increase in exercise). The compounds may already be approved for use in humans by an appropriate regulatory agency (e.g., the U.S. Food and Drug Administration or a foreign equivalent). The compounds may already be approved for use in humans for the treatment or prevention of atherogenesis, arteriosclerosis, atherosclerosis, or other cholesterol-related diseases. The compound can be any compound, molecule, polymer, macromolecule or molecular complex (e.g., proteins including biotherapeutics such as antibodies and enzymes, small organic molecules including known drugs and drug candidates, other types of small molecules, polysaccharides, fatty acids, vaccines, nucleic acids, etc) that can be screened for activity as outlined herein. Compounds are evaluated in the present invention for discovering potential therapeutic agents that affect cholesterol metabolism and transport. Compounds encompass numerous chemical classes. Compounds include known drugs or known drug agents or already-approved drugs. Known drugs also include, but are not limited to, any chemical compound or composition disclosed in, for example, the 13th Edition of The Merck Index (a U.S. publication, Whitehouse Station, N.J., USA), incorporated herein by reference in its entirety. The compounds may be proteins. The compounds may be naturally occurring proteins or fragments of naturally occurring proteins. The compounds may be antibodies or fragments thereof. The compounds may be nucleic acids.

In some embodiments, the invention provides methods comprising prognosing, diagnosing, and/or predicting a response to treatment of a condition associated with a deficiency in a reverse cholesterol transport (RCT) pathway in a subject the methods comprising the steps of (a) administering a subject with a modulator that specifically modulates a reverse cholesterol transporter pathway; (b) assessing lipid efflux profile, mRNA expression, protein expression, protein activation level and/or a phenotype in the at least one cell from the subject or a medium comprising said cell; (c) determining whether there is a deficiency in the reverse cholesterol transport pathway of the subject, where the determining is based in the assessing of lipid efflux profile, mRNA expression, protein expression, protein activation level and/or the phenotype in the at least one macrophage or macrophage-like cell; and (d) prognosing, diagnosing, and/or predicting a response to treatment of the condition associated with a deficiency in a reverse cholesterol transport (RCT) pathway, where the prognosing, diagnosing, and/or predicting a response to treatment is based in the determining in step (c). In some embodiments, the cell is a macrophage or a macrophage-like cell. The medium comprising the cell can be, for example, tissue, organ, blood, serum, plasma, body fluid, or culture media. In some embodiments, the reverse cholesterol transport modulator is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

In some embodiments, the invention provides methods for prognosing, diagnosing, and/or predicting a response to treatment of a condition associated with a deficiency in a reverse cholesterol transport (RCT) pathway in a subject the method comprising the steps of: (a) administering a subject with a modulator that specifically modulates a reverse cholesterol transporter pathway; (b) assessing the mobilization of a biomarker from tissue to plasma in the subject; and (c) prognosing, diagnosing, and/or predicting a response to treatment of the condition associated with a deficiency in a reverse cholesterol transport (RCT) pathway, where the prognosing, diagnosing, and/or predicting a response to treatment is based in the assessing in step (b). In some embodiments, the biomarker is a lipid, a protein or a nucleic acid as described herein. In some embodiments, the reverse cholesterol transport modulator is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

In some embodiments, the present invention provides a method of prognosing and/or diagnosing a subject with deficiency in the reverse cholesterol transport (RCT) pathway comprising: (a) isolating macrophage or a macrophage-like cell from the subject; (b) contacting the macrophage or macrophage-like cell with a compound that specifically modulates a reverse cholesterol transporter pathway; (c) assessing lipid efflux profile of the macrophage or macrophage-like cell treated with the compound as compared to lipid efflux profile of a control cell of the same type; and (d) determining whether there is a deficiency in the reverse cholesterol transport pathway of the subject. In some embodiments, the compound that specifically modulates the reverse cholesterol transporter pathway is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

In some embodiments, the present invention provides a method of predicting or identifying response of a subject with deficiency in reverse cholesterol transport (RCT) to treatment with a modulator of a reverse cholesterol transport pathway comprising: (a) isolating macrophage or a macrophage-like cell from the subject; (b) contacting the macrophage or macrophage-like cell with a modulator that is specific for a reverse cholesterol transporter pathway; (c) comparing lipid efflux profiles of the macrophage or macrophage-like cell treated with or without the modulator and (d) determining whether the subject responds to treatment of the modulator. In some embodiments, the reverse cholesterol transport modulator is a peptide that modulates an ATP-mediated transporter. In some embodiments, the ATP-mediated transporter is an ATP-binding cassette transporter (ABC-transporter). In some embodiments, the ABC transporter is ABC transporter sub-family A member 1 (ABCA1). In some embodiments, the ABC transporter is ABC transporter sub-family G member 1 (ABCG1) or ABCG8.

In some embodiments, responders to treatment are identified in two principal ways: (i) measuring defined lipids mobilization to plasma after treatment with a modulator specific for an RCT pathway or (ii) taking the subject plasma, spiking it with a mediator of specific efflux transporter—for example a ABCA1 selective peptide—, and assess the improve capacity compared to plasma alone to extract lipids from macrophage cells. A third way would be to collect monocytes from an individual, process the monocyte in vitro, and assess changes in the mRNA level of key regulatory genes by coincubation with for example a ABCA1 specific peptide.

In some embodiments, mRNA for the key regulatory genes and the lipid efflux from macrophages are assessed simultaneously or sequentially. By assessing intracellular changes in mRNA for the key regulatory genes and the lipid efflux from macrophages (e.g. from different individuals) the response to, for example, an ABCA1 specific peptide can be evaluated at both a gene and functional level.

In one example, a peptide compound which is selective, for example, for the ABCA1 transporter is co-incubated with a macrophage cell. In some embodiments, the profile of the peptide mediated lipid efflux is assessed, including but not limited to cholesterol and phospholipids contents. In some embodiments, the change in lipids contents of the cells is assessed. In some embodiments, in vivo change in plasma lipids content is assessed before, during and after treatment that stimulate ABCA1 transporter mediated efflux. In some embodiments, the mRNA changes induced by the selective ABCA1 treatment are assessed by standard methods, for example, PCR (Polymerase Chain Reaction). In some embodiments, a genotype specific pattern for the above described biology is assessed in an animal including but not limited to a human.

In one aspect, the present invention provides a method of modulating reverse cholesterol transport, the method comprising administering an effective amount of a peptide in a subject, wherein the peptide modulates activity of a reverse cholesterol transporter (RCT). In some embodiments, the RCT is ABCA1.

In some embodiments, the present invention provides methods for the treatment of a variety of RCT related diseases using a compound that modulates a RCT pathway, for example, a peptide that modulates the ABC transporters. In some embodiments, the present invention provides method for the treatment and diagnosis of RCT related diseases using a compound that modulates a RCT pathway, for example, a peptide that modulates the ABC transporters. Thus, the invention includes methods in which a patient is treated, diagnosed and a treatment outcome is predicted. For example, a patient might be diagnosed with a condition, given a treatment and after receiving the treatment obtained a prediction of outcome using the methods described herein. Thus, the invention provides companion diagnosis methods to treatment methods.

As used herein and as well understood in the art, examples of treatment include obtaining beneficial or desired results, including clinical results. As described herein, non-limiting examples of beneficial or desired clinical results include one or more of, but are not limited to, alleviation or amelioration of one or more symptoms, diminishment of extent of a condition, including a disease, stabilized (i.e., not worsening) state of a condition, including diseases, preventing spread of disease, delay or slowing of condition, including disease, progression, amelioration or palliation of the condition, including disease, state, and remission (whether partial or total), whether detectable or undetectable. In some variations, chlorite as described herein are used to achieve one or more of treating, preventing, delaying the onset of, or causing the regression of the diseases or conditions described herein.

The appropriate level of therapeutic agent for different subjects, including but not limited to a human subject, may be estimated there from using methods known by those of skill in the art. Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

The therapeutic agent, e.g., peptide that modulate ATP-transporter such as ATI-5261 or Y26 or peptide complexes as described herein, can be administered alone or as part of a combination therapy, e.g., administered in combination with or adjunctive to other common therapies for treating the diseases or conditions described herein. Administration of the compound may be prior to, subsequent to, or concurrent with one or more other treatments, including but not limited to treatments using other active agents or non-pharmaceutical therapies such as radiotherapy. In some variations the peptide compounds are used in accordance with their standard or common dosages, which can be determined by a skilled physician in the relevant field. The therapeutic agents may be administered by any suitable route of administration known in the art, for example, the subject peptides described herein may be administered by any of systemic, parenteral (e.g., intramuscular, intraperitoneal, intravenous, ICV, intracisternal injection or infusion, subcutaneous injection, or implant), by inhalation spray, nebulized or aerosolized using aerosol propellants, nasal, vaginal, rectal, sublingual, urethral (e.g., urethral suppository), by infusion, intraarterial, intrathecal, intrabronchial, subcutaneous, intradermal, intravenous, intracervical, intraabdominal, intracranial, intrapulmonary, intrathoracic, intratracheal, nasal routes, oral administration that delivers the therapeutic agent systemically, drug delivery device, or by a dermal patch that delivers the therapeutic agent systemically, transdermally or transbuccally. In some variations, the formulation is formulated for other than oral or transbuccal administration.

Kits

In some embodiments the invention provides kits. Kits provided by the invention may comprise one or more of the RCT pathway modulator described herein, such as ABC-transporter modulating peptides. A kit may also include other reagents that are useful in the invention, such as reagents to assess lipid efflux profile, mRNA expression, protein expression, protein activation level and/or a phenotype of a cell. A may also include a therapeutic agent. A kit may also include other reagents that are useful in the invention, such as fixatives, containers, plates, buffers, instructions, clinical data and the like.

The kit may be packaged in any suitable manner, typically with all elements in a single container along with a sheet of printed instructions for carrying out the test.

The kit may further comprise a software package for data analysis of the RCT pathway state, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The invention is further illustrated by the following non-limiting examples.

### EXAMPLES

### Example 1

Objective: Determine efficacy of a apoA-I mimetic, AT5261, in attenuation of lesion development in ApoE(−/−) flow cessation model.

AT5261 is a peptide optimized for selective and potent ABCA1 transporter mediated cholesterol efflux effect from macrophage foam cells. AT5261/Phospholipid complex is known to have a less selective ABCA1 effect. D4F is another peptide well described in the literature with a less selective ABCA1 effect than AT5261 free peptide.

Method: Following 2-week administration of high-fat diet to ApoE(−/−), and ligation of one carotid, AT5261 mimetic was administered at concentration 30 mg/kg, ip, every other day. Compound administration started on the day of surgery, prior to the procedure. High-fat diet feeding will continued through the length of the study, i.e., 15 days. Reference compound, D4F, 20 mg/kg, ip, every other day was used as a comparator. Control group was treated every other day, ip, with vehicle used to dissolve compounds, i.e., phosphate buffered saline at 5 mL/kg. Endpoints includes: cholesterol ester content of ligated artery as well as sphingolipids and phosphatidylcholine. Plasma lipids measured in the experiment include cholesterol, cholesterol ester, triglyceride, sphingolipids and phosphatidylcholine. Each group consisted of 12 animals.

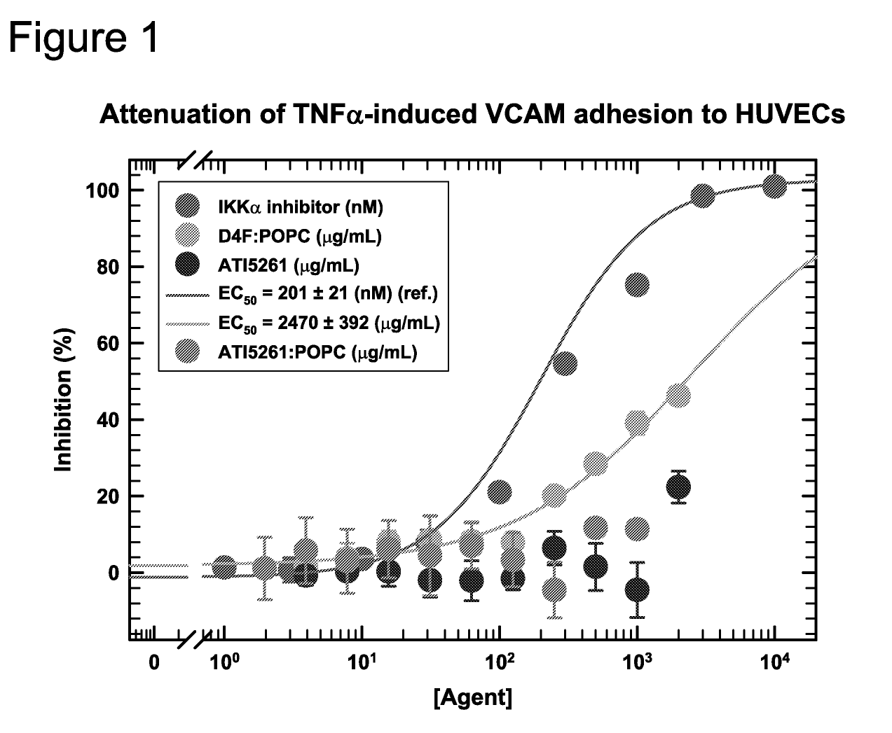

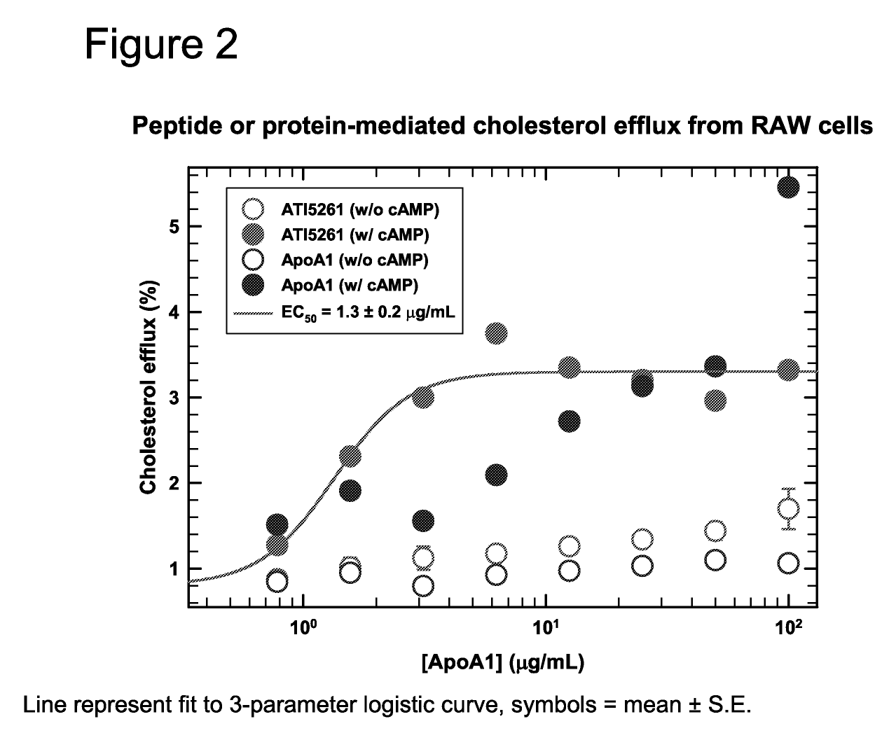

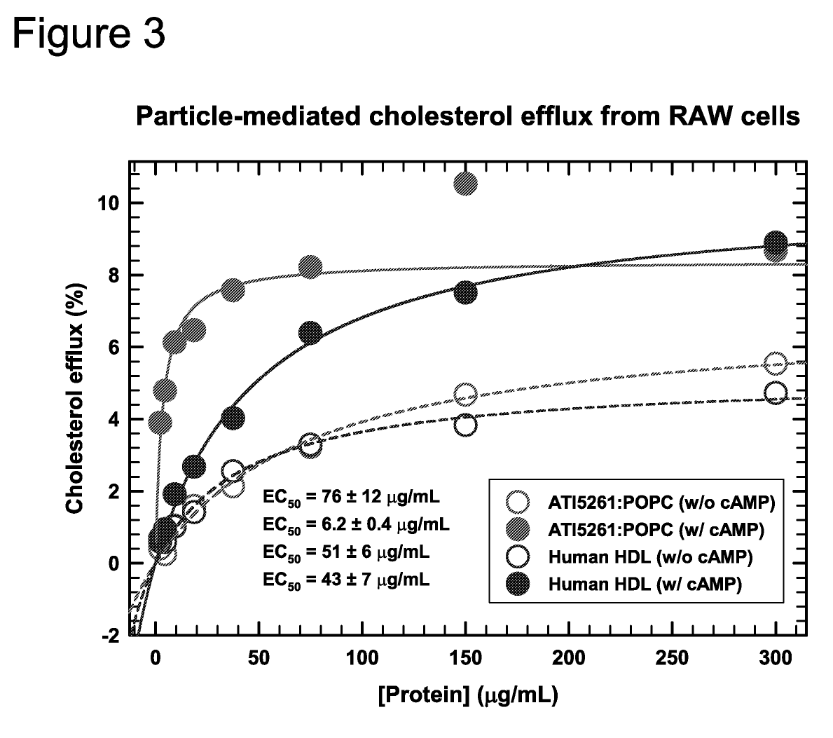

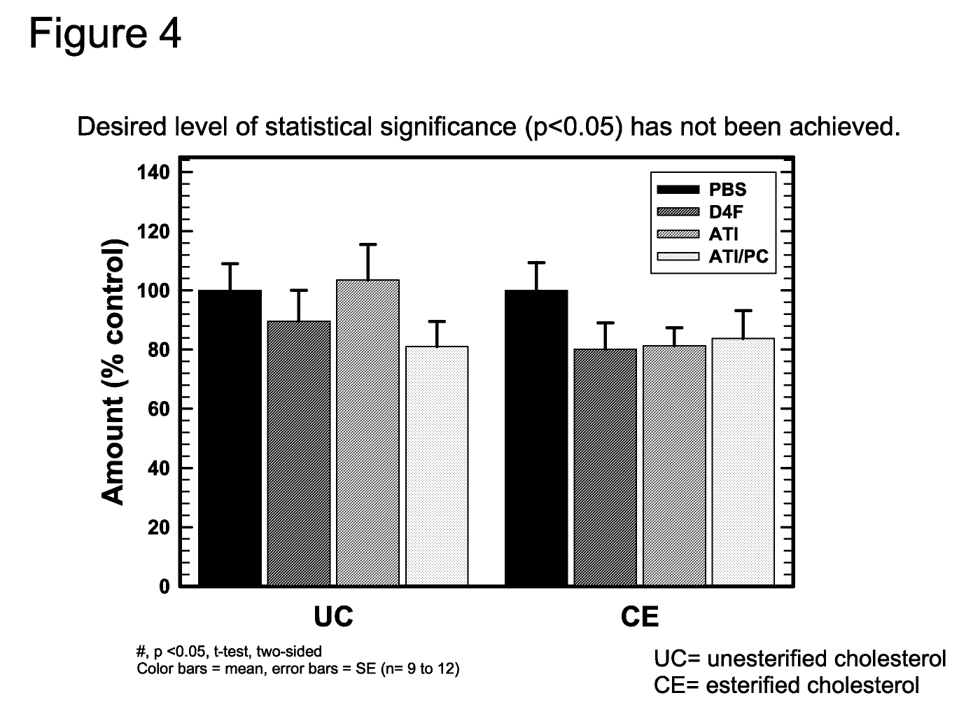

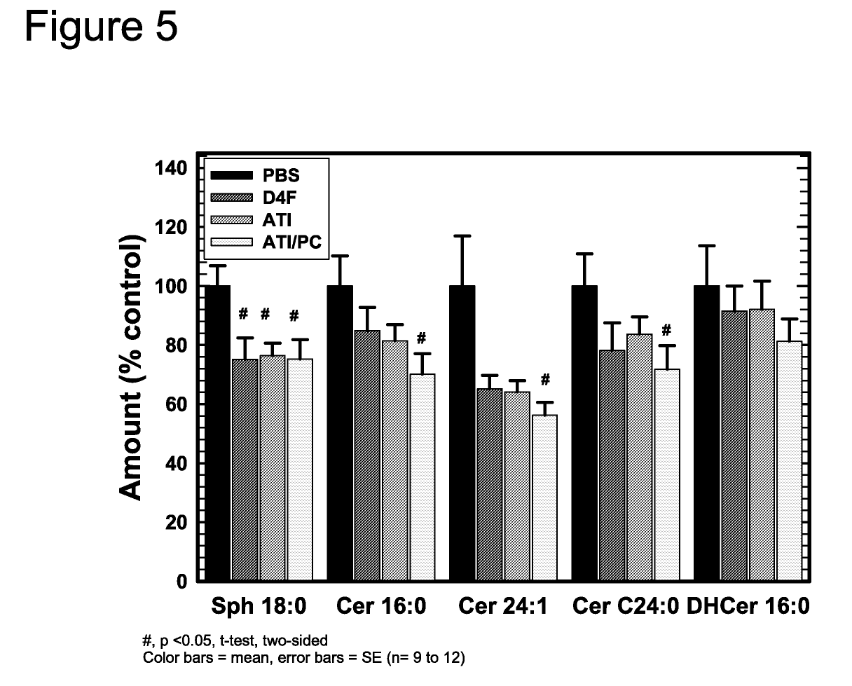

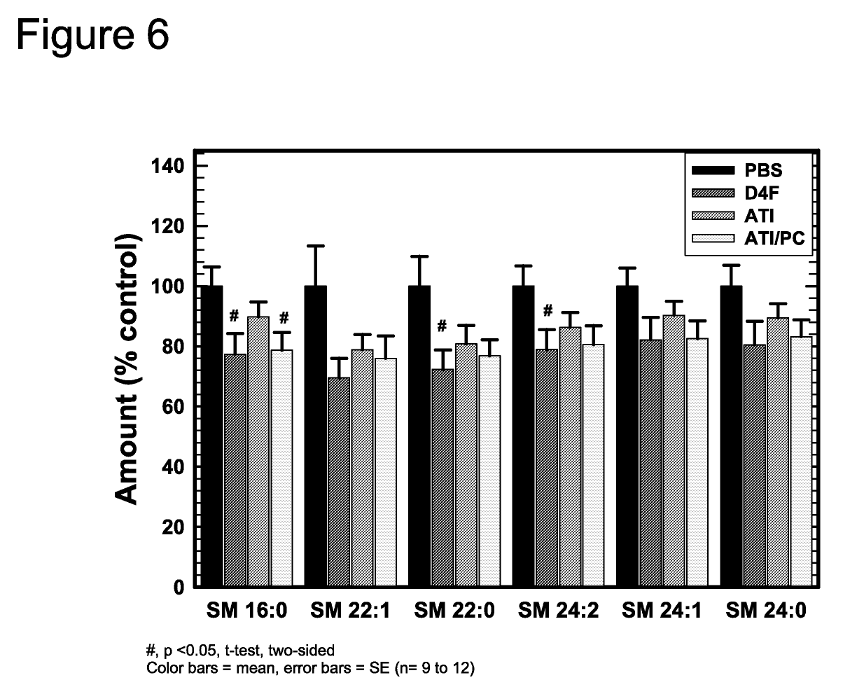

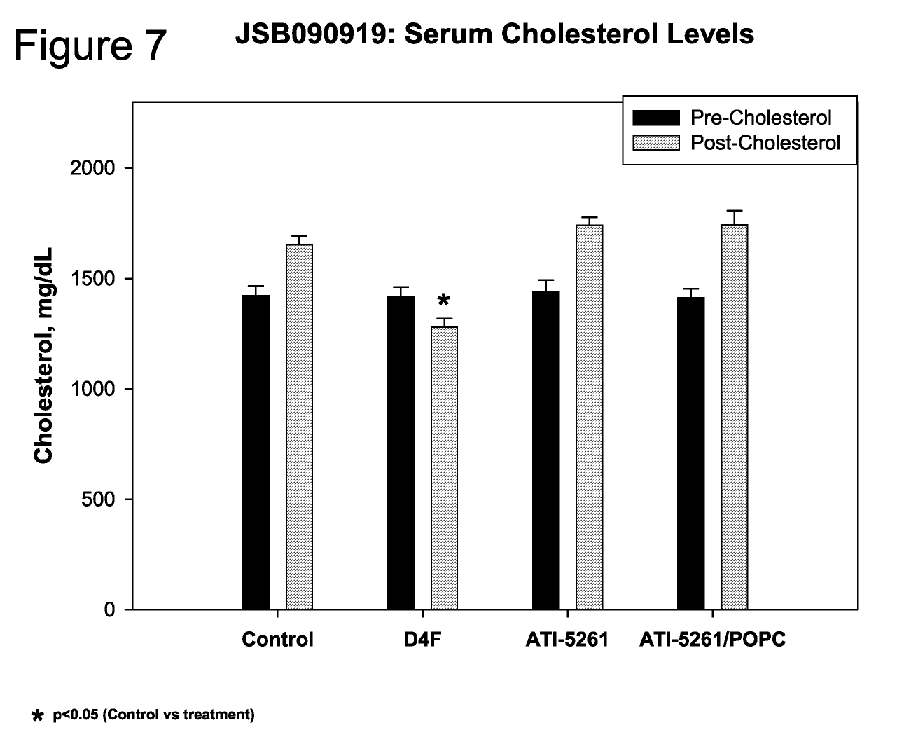

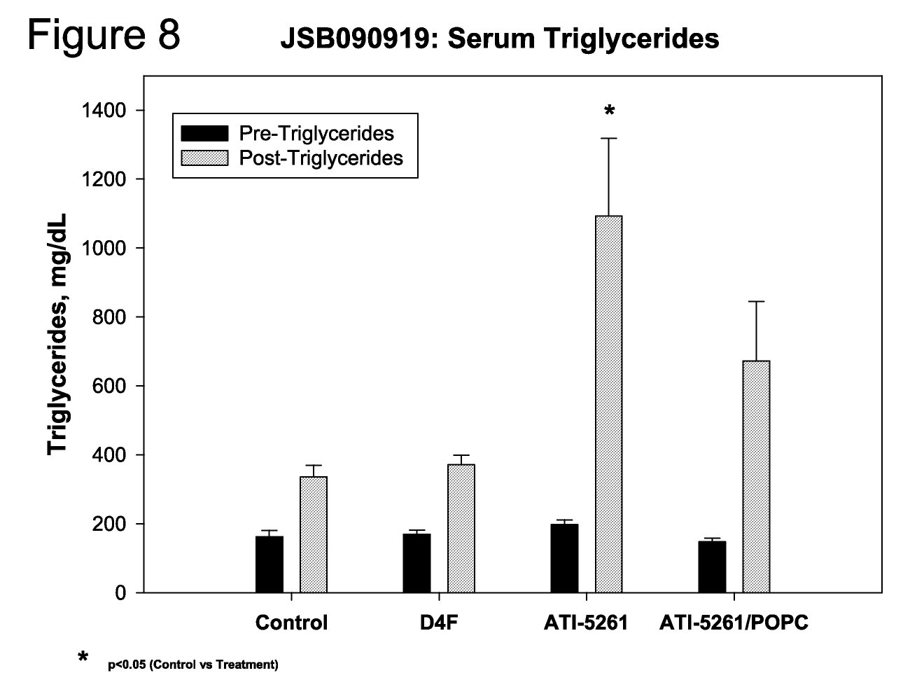

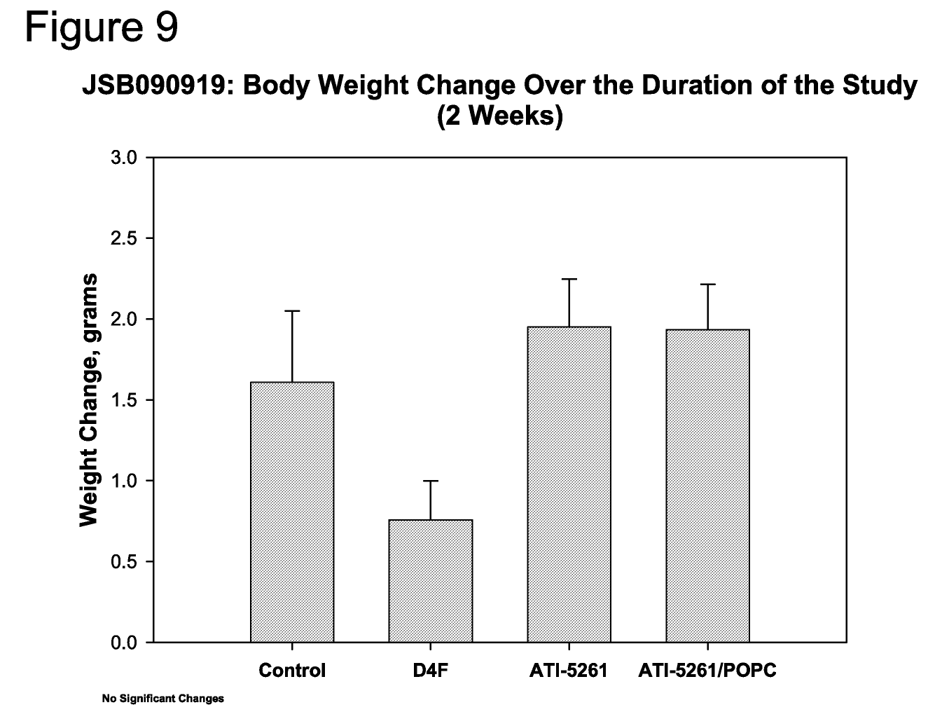

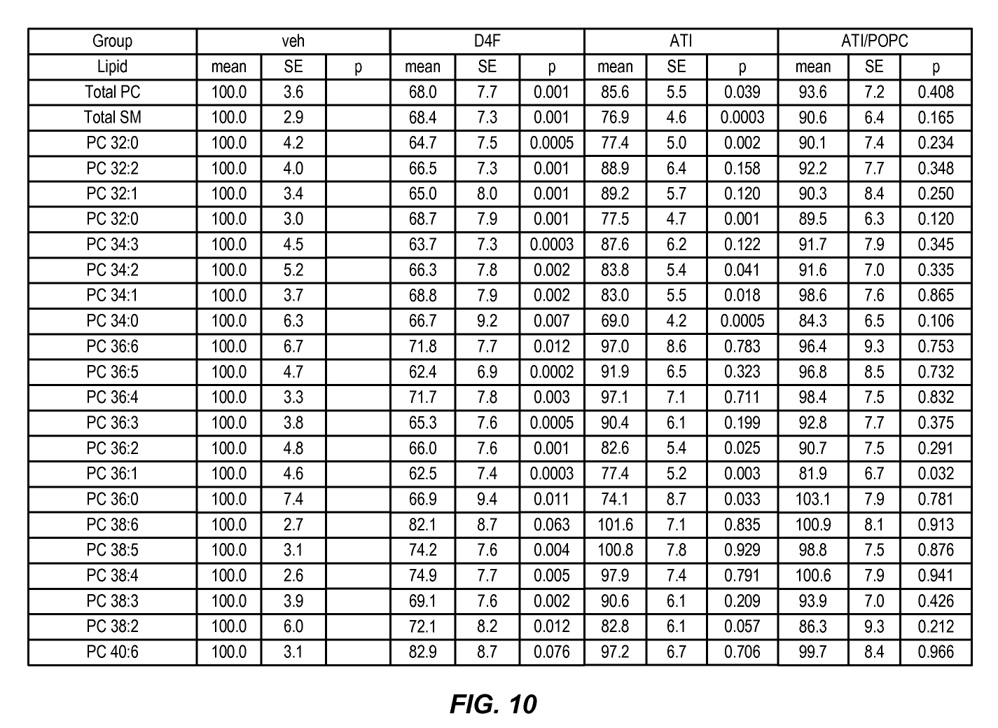

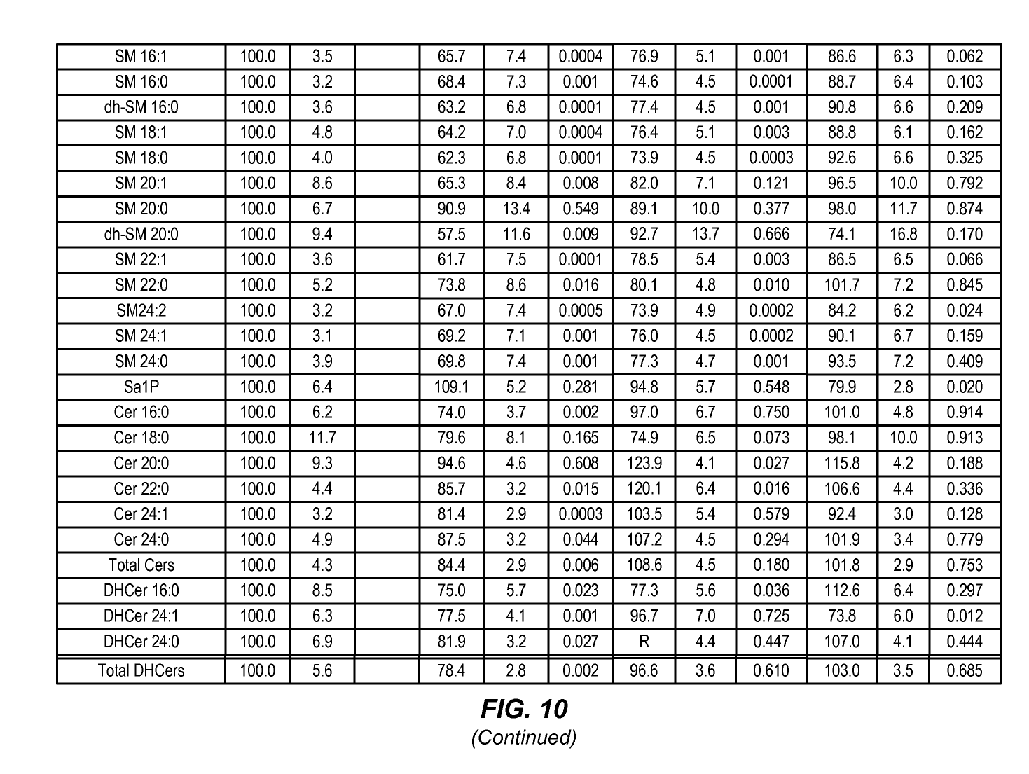

Results: Results are shown in FIGS. 1-10. AT5261 free peptide and AT5261/PL-complex both removed cholesterol ester and sphingomyelin from arteries with atherosclerosis, as do D4F, features known to be associated with histology evidence of atherosclerosis regression.

AT5261 free peptide as opposed to AT5261/PL complex was selective in lowering the concentrations of plasma Sphingomyelin (SM) and saturated Phosphatidyl Cholines. Each of treatment with D4F, AT5261 free peptide and AT5261/PL-complex resulted in different fingerprint patterns of plasma lipids reflecting different biological properties.

### Example 2

Purpose: Explore the effects of an Apo A1 mimetic in the ApoE mouse Flow Cessation Model.
I. Test Articles: Administered by ip injection

| | |
|---|---|
| D4F - LSN 2116424 (Lot# 0603-19) | Est. Qty. Needed: 72 mg |
| ATI-5261 (Lot# 208043-01) | Est. Qty. Needed: 144 mg |
| ATI-5261/POPC (Lot# 208043-01/8-27-09) | Est. Qty. Needed: 144 mg |

Vehicle for ip dosing will be PBS

Note: Dosing solution will be made by Dr. Marian Mosior 1 day before dosing.

### II. Animals:

ApoE −/− (male) Source: Taconic DOA=Sep. 1, 2009 Animal Use Protocol#3073

The mice will be approx. 9 week old at the start of the study (ligation surgery).

Mice will be maintained on Teklad 88137 (Western Diet) 2 weeks before ligation surgery and for the duration of the study.

Ligation surgery by Dr. Rekhter.

III. Groups: (12mice/group) Total of 48 mice.

| GROUP | DOSE | Dosing | Route | An# | n |
|---|---|---|---|---|---|
| a. Control (PBS) | — | Every other day | ip | A1-A12 | 12 |
| b. D4F | 20 mg/kg | Every other day | ip | B1-B12 | 12 |
| c. ATI-5261 | 40 mg/kg | Every other day | ip | C1-C12 | 12 |
| d. ATI-5261/POPC | 40 mg/kg | Every other day | iP | D1-D12 | 12 |

### IV. Dosing:

Test articles or vehicle will be dosed in the am(6:00-7:00) and with dosing volume of 0 ml/kg. First dose will be the day of ligation surgery, before the procedure. Last dose will be on the day of termination.

### V. Parameters

a. Body weights (day0 and termination) (A. Austin)
b. EDTA plasma for Cholesterol and Triglycerides (25 uI)—Pre-study for sort and termination (C. Reidy)
c. EDTA plasma (50 uL) for sphingomyelins, phosphatidylcholines, ceramides and sphingosine-1-phosphate (D. Peake, H. Bui) at termination of the study.
d. Tissue collection—Left Carotid artery for lipid extraction. Place carotid in 3 ml of 2:1 chloroform/methanol mix. Allow 16 hours for lipid extraction. (Cholesterol Ester analysis by M. Kalbfleisch) Share extract with David Peake and Hai Bui for measurements of sphingomyelins, phosphatidylcholines, ceramides and sphingosine-1-phosphate.
e. No Histology (save carotids in chlor/meth)

| Group | veh | | | D4F | | | ATI | | | ATI/POPC | | |
|---|---|---|---|---|---|---|---|---|---|---|---|---|
| Lipid | mean | SE | p | mean | SE | p | mean | SE | p | mean | SE | p |
| Total PC | 100.0 | 3.6 | | 68.0 | 7.7 | 0.001 | 85.6 | 5.5 | 0.039 | 93.6 | 7.2 | 0.408 |
| Total SM | 100.0 | 2.9 | | 68.4 | 7.3 | 0.001 | 76.9 | 4.6 | 0.0003 | 90.6 | 6.4 | 0.165 |
| PC 30:0 | 100.0 | 4.2 | | 64.7 | 7.5 | 0.0005 | 77.4 | 5.0 | 0.002 | 90.1 | 7.4 | 0.234 |
| PC 32:2 | 100.0 | 4.0 | | 66.5 | 7.3 | 0.001 | 88.9 | 6.4 | 0.158 | 92.2 | 7.7 | 0.348 |
| PC 32:1 | 100.0 | 3.4 | | 65.0 | 8.0 | 0.001 | 89.2 | 5.7 | 0.120 | 90.3 | 8.4 | 0.250 |
| PC 32:0 | 100.0 | 3.0 | | 68.7 | 7.9 | 0.001 | 77.5 | 4.7 | 0.001 | 89.5 | 6.3 | 0.120 |
| PC 34:3 | 100.0 | 4.5 | | 63.7 | 7.3 | 0.0003 | 87.6 | 6.2 | 0.122 | 91.7 | 7.9 | 0.345 |
| PC 34:2 | 100.0 | 5.2 | | 66.3 | 7.8 | 0.002 | 83.8 | 5.4 | 0.041 | 91.6 | 7.0 | 0.335 |
| PC 34:1 | 100.0 | 3.7 | | 68.8 | 7.9 | 0.002 | 83.0 | 5.5 | 0.018 | 98.6 | 7.6 | 0.865 |
| PC 34:0 | 100.0 | 6.3 | | 66.7 | 9.2 | 0.007 | 69.0 | 4.2 | 0.0005 | 84.3 | 6.5 | 0.106 |
| PC 36:6 | 100.0 | 6.7 | | 71.8 | 7.7 | 0.012 | 97.0 | 8.6 | 0.783 | 96.4 | 9.3 | 0.753 |
| PC 36:5 | 100.0 | 4.7 | | 62.4 | 6.9 | 0.0002 | 91.9 | 6.5 | 0.323 | 96.8 | 8.5 | 0.732 |
| PC 36:4 | 100.0 | 3.3 | | 71.7 | 7.8 | 0.003 | 97.1 | 7.1 | 0.711 | 98.4 | 7.5 | 0.832 |
| PC 36:3 | 100.0 | 3.8 | | 65.3 | 7.6 | 0.0005 | 90.4 | 6.1 | 0.199 | 92.8 | 7.7 | 0.375 |
| PC 36:2 | 100.0 | 4.8 | | 66.0 | 7.6 | 0.001 | 82.6 | 5.4 | 0.025 | 90.7 | 7.5 | 0.291 |
| PC 36:1 | 100.0 | 4.6 | | 62.5 | 7.4 | 0.0003 | 77.4 | 5.2 | 0.003 | 81.9 | 6.7 | 0.032 |
| PC 36:0 | 100.0 | 7.4 | | 66.9 | 9.4 | 0.011 | 74.1 | 8.7 | 0.033 | 103.1 | 7.9 | 0.781 |
| PC 38:6 | 100.0 | 2.7 | | 82.1 | 8.7 | 0.063 | 101.6 | 7.1 | 0.835 | 100.9 | 8.1 | 0.913 |
| PC 38:5 | 100.0 | 3.1 | | 74.2 | 7.6 | 0.004 | 100.8 | 7.8 | 0.929 | 98.8 | 7.5 | 0.876 |
| PC 38:4 | 100.0 | 2.6 | | 74.9 | 7.7 | 0.005 | 97.9 | 7.4 | 0.791 | 100.6 | 7.9 | 0.941 |
| PC 38:3 | 100.0 | 3.9 | | 69.1 | 7.6 | 0.002 | 90.6 | 6.1 | 0.209 | 93.9 | 7.0 | 0.426 |
| PC 38:2 | 100.0 | 6.0 | | 72.1 | 8.2 | 0.012 | 82.8 | 6.1 | 0.057 | 86.3 | 9.3 | 0.212 |
| PC 40:6 | 100.0 | 3.1 | | 82.9 | 8.7 | 0.076 | 97.2 | 6.7 | 0.706 | 99.7 | 8.4 | 0.966 |
| SM 16:1 | 100.0 | 3.5 | | 65.7 | 7.4 | 0.0004 | 76.9 | 5.1 | 0.001 | 86.6 | 6.3 | 0.062 |
| SM 16:0 | 100.0 | 3.2 | | 68.4 | 7.3 | 0.001 | 74.6 | 4.5 | 0.0001 | 88.7 | 6.4 | 0.103 |
| dh-SM 16:0 | 100.0 | 3.6 | | 63.2 | 6.8 | 0.0001 | 77.4 | 4.5 | 0.001 | 90.8 | 6.6 | 0.209 |
| SM 18:1 | 100.0 | 4.8 | | 64.2 | 7.0 | 0.0004 | 76.4 | 5.1 | 0.003 | 88.8 | 6.1 | 0.162 |
| SM 18:0 | 100.0 | 4.0 | | 62.3 | 6.8 | 0.0001 | 73.9 | 4.5 | 0.0003 | 92.6 | 6.6 | 0.325 |
| SM 20:1 | 100.0 | 8.6 | | 65.3 | 8.4 | 0.008 | 82.0 | 7.1 | 0.121 | 96.5 | 10.0 | 0.792 |
| SM 20:0 | 100.0 | 6.7 | | 90.9 | 13.4 | 0.549 | 89.1 | 10.0 | 0.377 | 98.0 | 11.7 | 0.874 |
| dh-SM 20:0 | 100.0 | 9.4 | | 57.5 | 11.6 | 0.009 | 92.7 | 13.7 | 0.666 | 74.1 | 16.8 | 0.170 |
| SM 22:1 | 100.0 | 3.6 | | 61.7 | 7.5 | 0.0001 | 78.5 | 5.4 | 0.003 | 86.5 | 6.5 | 0.066 |
| SM 22:0 | 100.0 | 5.2 | | 73.8 | 8.6 | 0.016 | 80.1 | 4.8 | 0.010 | 101.7 | 7.2 | 0.845 |
| SM 24:2 | 100.0 | 3.2 | | 67.0 | 7.4 | 0.0005 | 73.9 | 4.9 | 0.0002 | 84.2 | 6.2 | 0.024 |
| SM 24:1 | 100.0 | 3.1 | | 69.2 | 7.1 | 0.001 | 76.0 | 4.5 | 0.0002 | 90.1 | 6.7 | 0.159 |
| SM 24:0 | 100.0 | 3.9 | | 69.8 | 7.4 | 0.001 | 77.3 | 4.7 | 0.001 | 93.5 | 7.2 | 0.409 |
| Sa1P | 100.0 | 6.4 | | 109.1 | 5.2 | 0.281 | 94.8 | 5.7 | 0.548 | 79.9 | 2.8 | 0.020 |
| Cer 16:0 | 100.0 | 6.2 | | 74.0 | 3.7 | 0.002 | 97.0 | 6.7 | 0.750 | 101.0 | 4.8 | 0.914 |
| Cer 18:0 | 100.0 | 11.7 | | 79.6 | 8.1 | 0.165 | 74.9 | 6.5 | 0.073 | 98.1 | 10.1 | 0.913 |
| Cer 20:0 | 100.0 | 9.3 | | 94.6 | 4.6 | 0.608 | 123.9 | 4.1 | 0.027 | 115.8 | 4.2 | 0.188 |
| Cer 22:0 | 100.0 | 4.4 | | 85.7 | 3.2 | 0.015 | 120.1 | 6.4 | 0.016 | 106.6 | 4.4 | 0.336 |
| Cer 24:1 | 100.0 | 3.2 | | 81.4 | 2.9 | 0.0003 | 103.5 | 5.4 | 0.579 | 92.4 | 3.0 | 0.128 |
| Cer 24:0 | 100.0 | 4.9 | | 87.5 | 3.2 | 0.044 | 107.2 | 4.5 | 0.294 | 101.9 | 3.4 | 0.779 |
| Total Cers | 100.0 | 4.3 | | 84.4 | 2.9 | 0.006 | 108.6 | 4.5 | 0.180 | 101.8 | 2.9 | 0.753 |
| DHCer 16:0 | 100.0 | 8.5 | | 75.0 | 5.7 | 0.023 | 77.3 | 5.6 | 0.036 | 112.6 | 6.4 | 0.297 |
| DHCer 24:1 | 100.0 | 6.3 | | 72.5 | 4.1 | 0.001 | 96.7 | 7.0 | 0.725 | 73.8 | 6.0 | 0.012 |
| DHCer 24:0 | 100.0 | 6.9 | | 81.9 | 3.2 | 0.027 | 106.3 | 4.4 | 0.447 | 107.0 | 4.1 | 0.444 |
| Total DHCers | 100.0 | 5.6 | | 78.4 | 2.8 | 0.002 | 96.6 | 3.6 | 0.610 | 103.0 | 3.5 | 0.685 |

### Example 3

ApoE KO mice that received HFWD (high fat western diet) and had ligations of their carotid artery (the so called flow cessation model also described in Example 1) were treated with AT5261 30 mg/kg in alternate days for 14 days. At termination, following 2 weeks treatment, the carotid vessel tissue was analyzed for lipid content by LC-MS/MS. Compared to vehicle control the AT5261 treated mice showed approximately 20% lowering of cholesterol ester. AT5261 lowered sphingolipids contents (spingosine, ceramide, sphoingomyelin) approximately 20%. Spingosine 18:0/So(18) Carotid Artery tissue concentration was lowered by 23.6% (p=0.009) by free AT5261 treatment. Table 2 below shows a summary of the results.

TABLE 2

| Group | veh | | | D4F | | | ATI | | | ATI/POPC | | |
|---|---|---|---|---|---|---|---|---|---|---|---|---|
| Lipid | mean | SE | p | mean | SE | p | mean | SE | p | mean | SE | p |
| Sph 18:0 | 100.0 | 6.9 | | 75.2 | 7.3 | 0.024 | 76.4 | 4.2 | 0.009 | 75.3 | 6.6 | 0.023 |
| Cer 16:0 | 100.0 | 10.2 | | 84.9 | 7.9 | 0.262 | 81.4 | 5.5 | 0.127 | 70.1 | 7.0 | 0.039 |
| Cer 24:1 | 100.0 | 16.9 | | 65.1 | 4.6 | 0.060 | 64.1 | 3.9 | 0.051 | 56.2 | 4.3 | 0.042 |
| Cer C24:0 | 100.0 | 11.0 | | 78.2 | 9.3 | 0.152 | 83.7 | 5.8 | 0.206 | 71.7 | 8.1 | 0.069 |
| DHCer 16:0 | 100.0 | 13.6 | | 91.5 | 8.5 | 0.607 | 92.0 | 9.6 | 0.644 | 81.2 | 7.6 | 0.291 |
| PC 34:2 | 100.0 | 8.7 | | 75.9 | 9.6 | 0.076 | 88.4 | 5.9 | 0.279 | 80.6 | 8.8 | 0.141 |
| PC 34:1 | 100.0 | 7.7 | | 77.7 | 8.8 | 0.070 | 94.1 | 6.6 | 0.568 | 82.9 | 9.7 | 0.177 |
| PC 36:4 | 100.0 | 6.0 | | 88.7 | 9.6 | 0.330 | 96.1 | 5.9 | 0.647 | 85.7 | 8.7 | 0.180 |
| PC 36:3 | 100.0 | 7.4 | | 79.4 | 10.1 | 0.114 | 98.6 | 6.9 | 0.891 | 87.7 | 9.8 | 0.320 |
| PC 36:2 | 100.0 | 8.3 | | 78.9 | 9.8 | 0.115 | 92.8 | 6.8 | 0.508 | 83.1 | 9.9 | 0.206 |
| PC 36:1 | 100.0 | 9.4 | | 79.4 | 9.0 | 0.128 | 95.2 | 6.8 | 0.687 | 81.6 | 9.8 | 0.198 |
| PC 38:6 | 100.0 | 6.5 | | 90.9 | 10.5 | 0.472 | 97.6 | 6.1 | 0.793 | 87.0 | 8.7 | 0.236 |
| PC 38:5 | 100.0 | 5.2 | | 88.4 | 9.8 | 0.307 | 106.2 | 7.3 | 0.495 | 88.3 | 10.4 | 0.294 |
| PC 38:4 | 100.0 | 6.1 | | 93.2 | 10.5 | 0.578 | 103.6 | 7.3 | 0.705 | 85.1 | 9.1 | 0.175 |
| SM 16:0 | 100.0 | 6.3 | | 77.4 | 6.8 | 0.024 | 89.8 | 4.9 | 0.215 | 78.7 | 5.8 | 0.027 |
| SM 22:1 | 100.0 | 13.3 | | 69.5 | 6.5 | 0.051 | 78.9 | 5.1 | 0.152 | 76.0 | 7.4 | 0.167 |
| SM 22:0 | 100.0 | 9.8 | | 72.3 | 6.4 | 0.028 | 80.8 | 6.1 | 0.112 | 76.8 | 5.3 | 0.075 |
| SM 24:2 | 100.0 | 6.7 | | 79.0 | 6.6 | 0.036 | 86.2 | 4.9 | 0.113 | 80.5 | 6.3 | 0.055 |
| SM 24:1 | 100.0 | 6.0 | | 82.1 | 7.5 | 0.075 | 90.3 | 4.6 | 0.210 | 82.5 | 5.9 | 0.055 |
| SM 24:0 | 100.0 | 6.9 | | 80.5 | 7.8 | 0.075 | 89.4 | 4.7 | 0.218 | 83.1 | 5.6 | 0.088 |

### Example 4

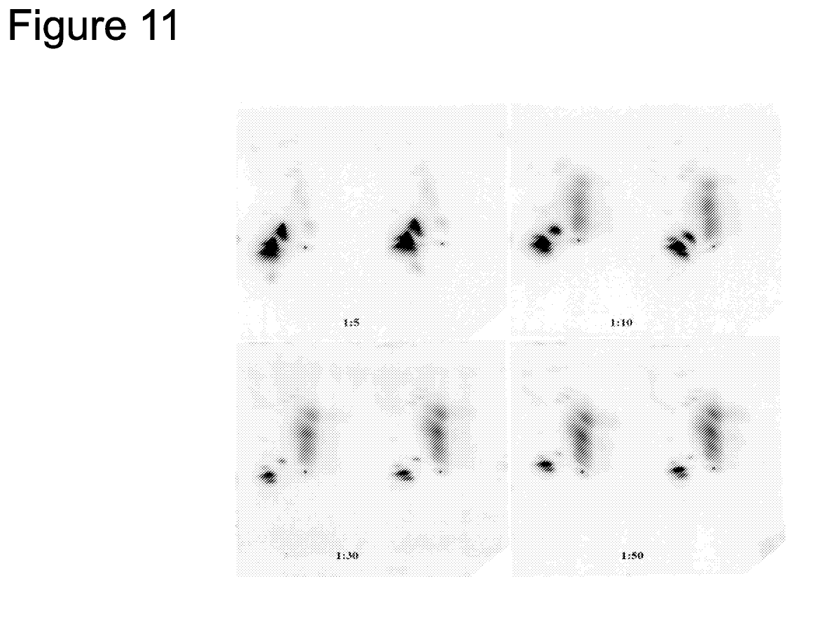

FIG. 11 is representative of findings that AT5261 Peptide Converts α-mobility HDL particles to preβ1-HDL. These results were obtained after incubating a normal human (female) plasma samples with peptide AT5261 in different dilutions. The incubations were 5 minutes at room temperature and mass concentrations were calculated for peptide: apoA-I as 1:5, 1:10, 1:30, and 1:50 by mass. The peptide in 1:50 ratio was still able to relocate apoA-I from the larger α-mobility particles to the small preβ-1 fraction. It is worth to mention that the peptide relocate apoA-I from only the α-mobility particles. Prep-2 is large, contains relatively large amount of phospholipids but not neutral core lipids. The peptide had no influence on preβ-2. Given this data it is likely that the formed small preβ-1 fraction is ideal in mediating ABCA1 mediated lipid efflux.

In a similar incubation experiment the change in HDL subclass concentrations were quantified with regard to apoA-I concentration in each subclass. For example, with incubation peptide:apoA-I mass ratios 1:100 the preβ1-HDL fraction increased from 5.46% to 22.12% of HDL, representing a relative 4-fold (405%) increase. Table 3 summarizes these results.

TABLE 3

| | Original | 1:100 (5 min) | | 1:50 (5 mm) | | 1:50 (60 min) | |
|---|---|---|---|---|---|---|---|
| Preβ-1 | 5.46 | 22.12 | (405%) | 35.87 | (657%) | 38.73 | (709%) |
| Preβ-2 | 1.16 | 0.45 | (38%) | 0.21 | (18%) | 0.12 | (10%) |
| α-1 | 19.11 | 14.06 | (74%) | 11.27 | (60%) | 10.27 | (54%) |
| α-2 | 38.61 | 30.79 | (80%) | 26.81 | (69%) | 24.42 | (63%) |
| α-3 | 18.15 | 15.80 | (87%) | 13.73 | (76%) | 14.26 | (79%) |
| α-4 | 6.18 | 8.12 | (131% | 7.11 | (115%) | 8.04 | (99%) |
| Preα-1 | 4.83 | 4.19 | (87%) | 2.20 | (46%) | 1.70 | (41%) |
| Preα-2 | 4.15 | 2.49 | (60%) | 1.67 | (40%) | 1.37 | (55%) |
| Preα-3 | 1.54 | 1.38 | (89%) | 0.65 | (42%) | 0.69 | (50%) |
| Preα-4 | 0.81 | 0.79 | (97%) | 0.40 | (50%) | 0.40 | (50%) |

### Example 5

AT5261 and Y26 peptide was administered ip to C57B mice following the dose regimen depicted in Table 4.

TABLE 4

| | |
|---|---|
| C5786 mice | Control, n = 4 |
| 5 ip | AT5261 51 mg/kg/48 h, n = 4 |
| Administration | AT5261 101 mg/kg/48 h, n = 4 |
| | Y26 101 mg/kg/48 h, n = 4 |
| | AT5261 5 mg/kg/24 h, n = 4 |
| | AT5261 51 mg/kg/24 h, n = 4 |
| | AT5261 101 mg/kg/24 h, n = 4 |

AT5261 and Y26 peptide treatment in C57B6 mice showed consistent decreases of plasma concentrations of acute phase reactants. Thus we observed treatment induced statistically significant decreases in CRP, Fibrinogen, Haptoglobin, IL-18, SAP (serum amyloid P component), RANTES, TIMP-1 and VCAM-1. The treatment effect followed a dose-response pattern, i.e. was more pronounced with higher doses and shorter dosing intervals.

Already at 5 mg/kg/24 h statistically significant decreases of plasma levels of CRP, fibrinogen, haptoglobin, IL-18 and SAP were found averaging approximately 25% across the acute phase reactant variables.

Differences between peptide treatments were discerned. Comparing alternate day treatment regimens Y26 treatment but not AT5261 treatment resulted in statistically significant decreases in MIP-1beta, MPO, and VEGF-alpha. In contrast AT5261 treatment but not Y26 treatment resulted in significant decreases in IL-7.

Both peptides show significant decreases in serum concentrations of acute phase reactants. More inflammation variables were decreased by Y26 suggesting that Y26 is a more potent anti-inflammatory compound than AT5261.

These results show that individual and/or combined plasma protein variables and their response to treatment algorithms can be developed to help assess treatment effect and predict treatment outcome.

### Example 6

### Lipidomics Assessment of AT5261 and Y26 Peptides in C57B6 Mice

The primary objective of the study was to analyze AT5261 properties with regard to lipid efflux properties. This was studied in a step wise fashion. First, efflux in vitro was compared from transformed macrophage cells (J774) (Study 1, see Table 5) by apolipoprotein A-I (apoA-I), natures strongest effluxer and AT5261 peptide, which has been optimized for ABCA1 mediated cholesterol efflux and shown to be about 5 fold more potent compared to apoA-I with regard to EC50. Secondly, mobilization of lipids to serum in vivo was assessed. C57B6 mice (Study 2, see Table 5) different treatment regimens of AT5261 in treatment 5-10 days long were applied. In this study AT5261 at one dose regimen was also compared to the same dose regimen of Y26, which is another peptide. Thirdly, in apoE KO mice made atherosclerotic by high fat cholesterol diet, (Study 3, see Table 5) the serum lipid profile following 42 days of treatment was compared to vehicle control with AT5261 given at two different doses. The objective of the experiment was to detect lipid variables that would have potential to become biomarkers of reverse lipid transport (RLT) and companion diagnostics to AT5261 treatment. The experimental design is shown in Table 5.

TABLE 5

| | Model and Readouts | Peptide and Administration |
|---|---|---|
| Study 1 | In vitro-J774 cells | AT5261, n = 2 |
| | (PCs, LPC, SL) | ApoA-I, n = 2 |
| Study 2 | C57B6 mice 5 ip Admin | Control, n = 4 |
| | (PCs, LPC, SL) | AT5261 51 mg/kg/48 h, n = 4 |
| | | AT5261 101 mg/kg/48 h, n = 4 |
| | | Y26 101 mg/kg/48 h, n = 4 |
| | | AT5261 5 mg/kg/24 h, n = 4 |
| | | AT5261 51 mg/kg/24 h, n = 4 |
| | | AT5261 101 mg/kg/24 h, n = 4 |
| Study 3 | ApoE KO HFD, 6 wks ip | Control, n = 11 |
| | (PCs, LPC, SL) | AT5261 5 mg/kg/24 h, n = 15 |
| | | AT5261 5 mg/kg/48 h, n = 15 |

Methods

Study 1: Isolation of Nascent HDL from Incubations of Macrophage Foam-Cells with AT5261 and apoA-I.

A commercial transformed macrophage cell-line, J774 (mouse), was used to evaluate lipid efflux responses to peptide ATI-5261 and ApoA-I. Macrophage foam-cells were prepared and exposed to lipid-free AT5261 peptide and ApoA-I, respectively; Nascent HDL products resulting from stimulation of cholesterol efflux were isolated and concentrated from conditioned medium and analyzed for lipid content.

Near confluent T175-flasks (30 total) of J774 mouse macrophages were incubated (2 days) with 0.1 mg/ml acetylated LDL in RPMI-1620 culture medium containing 1% FBS, to produce foam-cells. On the day of the experiment, cell monolayers were rinsed (3 times) with 20 ml/flask of HBSS, incubated 1 h with 0.2% BSA in serum-free RPMI-1640 medium, then rinsed (3 times) with serum-free RPMI. Rinsed cells were then exposed (20 ml/flask) to either apoA-I (15 flasks) or ATI-5261 (15 flasks), at a final concentration of 10 microgram/ml serum free RPMI. Following 20 h, conditioned medium was recovered from flasks, pooled within groups, and filtered (0.2 mμ). The pooled medium (˜300 ml) was subsequently concentrated to 5 ml using an Amicon stir-cell (10 K membrane) and density adjusted to 1.21 g/ml with solid NaBr. Nascent HDL was isolated by ultracentrifugation (120K, 5 h 20 minutes, 10° C.) using a TL-120 centrifuge, samples dialyzed to saline (pH=7.4) and filter sterilized (0.2 nip, Nalgene capsules). Isolated material was used for detailed MS of PL subclasses, subjected to non-denaturing gradient gel electrophoresis and protein lipid ratios determined by 280 abs. and commercial kits.

Lipid Extraction for LC-MS/MS

Samples were kept at −80° C. until extraction. Samples were allowed to thaw on ice and a small aliquot of each sample was transferred into siliconized glass tubes followed by addition of ultrapure water. Samples were spiked with either a mixture of C17-based ceramide, sphingosine, sphingosine-1-phosphate and sphingomyelin (10 ng of each as internal sphingolipid standards) or (1 mg of deuterated 16:0/16:0 PC as internal phosphatidylcholine standard). Samples were subjected to a Bligh and Dyer extraction (Bligh, E. G., Dryer, W. J. (1959) Can J Biochem physiol 37, 911-917). Lipid extracts were dried under stream of nitrogen at room temperature. The dried lipid extracts were then reconstituted in 100 ml ethanol/formic acid (99.8/0.2) and kept at 4° C. until LC-MS/MS analysis. Extracts were injected onto a reverse phase C18 HPLC Column to separate and resolve the various lipids. Phosphatidylcholine and sphingolipids were eluted off the column and analyzed on a triple quadrupole mass spectrometer Sciex API 5500. The data acquisition was performed in a targeted MRM (MS/MS) mode using lipid specific precursor ion to product ion (or parent to daughter ion) mass transitions resulting in highly sensitive and specific quantitative data. The resulting data was processed using Applied Biosystems Analyst 1.4.2 software.

Assessing total cholesterol (C) mobilization and total phospholipid (PL) mobilization by AT5261 and ApoA-I to media showed a constant C/PL ratio. Thus, the a Cholesterol: Phospholipid molar ratio was 2.74 (S.D. 0.40) for AT5261 and 2.89 (S.D. 0.68) for ApoA-I. However, AT5261 mobilized approximately twice as much lipid from cella to media compared to apoA-I. As described below the specific PLs species mobilized to media by AT5261 and apoA-I differed and are predicted to explain the higher lipid efflux capacity by AT5261.

Results Study 1, LC-MS/MS Phosphatidylcholin (PC) Analysis

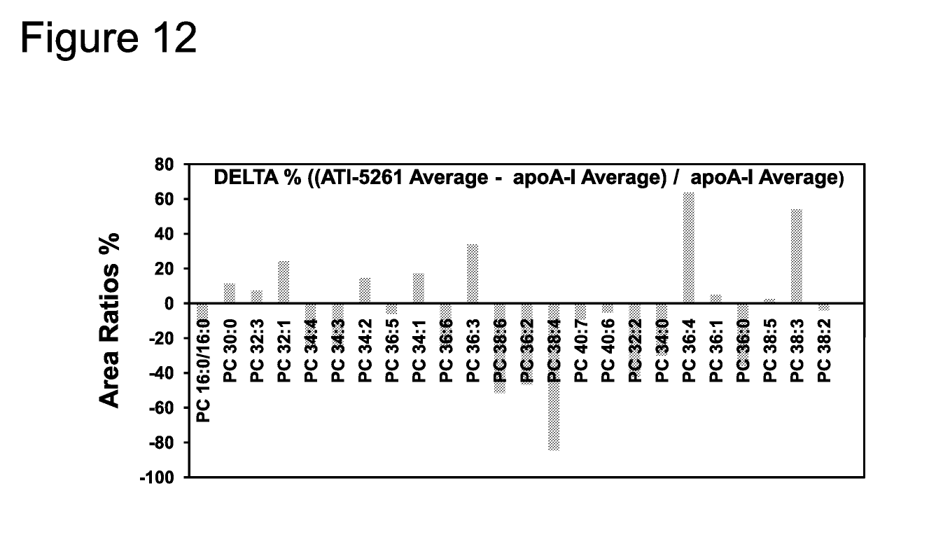

All phospholipid (PL) media concentrations increased by incubation with apoA-I and AT5261. A main finding is that AT5261 mobilized approximately twice as much PC to media (42 vs. 19). The values in FIG. 12 have been adjusted for that approximately 2-fold difference in order to allow a comparison of the type of PC that are being effluxed by apoA-I and AT5261, respectively. Extraction of various PCs from cells to media differed between AT5261 and apoA-I, for example AT5261 mobilized 40% and 60% more PC 36:3 and PC 36:4 while mobilization of PC 38:4 was considerably less by AT5261 than for ApoA-I. The data shows that this method can be used to discover and assess effects of new drugs with regard to lipid mobilizing properties from cells. It is also anticipated that cells from different sources would provide unique PC response profiles that could be used to assess that individuals reverse lipid transport propensity and thereby CVD risk.

Results Study 1, LC-MS/MS Lyso-Phosphatidylcholins (LPC)

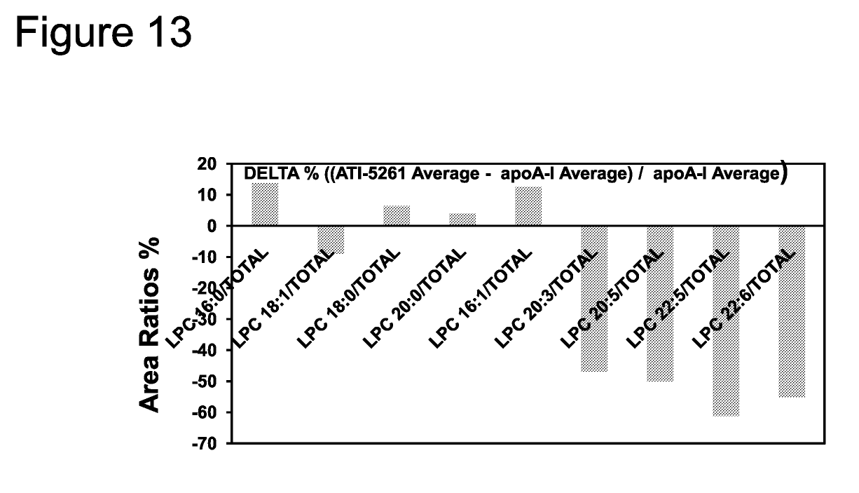

All LPC media concentrations increased by incubation with apoA-I and AT5261 A main finding is that AT5261 mobilized approx. twice as much LPC to media (81 vs. 40). The values in FIG. 13 have been adjusted for that 2-fold difference in order to allow a comparison of the type of LPC that are being effluxed by apoA-I and AT5261. Notably AT5261 mobilizes more of shorter chain and saturated LPC which contrasts to apoA-I which mobilizes more of longer chain and more saturated LPCs. Each group had an n=2 not allowing statistical analysis. The data shows that the method can be used in discovery to assess RCT fingerprint properties of peptides. By having different sources of macrophages, for example from one patient that will develop CVD and one that will not, this method can be used for predicting CVD risk. Also, effect by treatment can be assessed following change in phospholipid efflux over time, thereby optimizing treatment to reach wanted treatment effect on for example cardiovascular disease.

Results Study 1, LC-MS/MS Sphingomyelin (SM)

AT5261 effluxed more SMs than ApoA-I to the media, this difference was particularly pronounced for DiHy Ceramide 18:1.

In summary assessments of PC, LPC and sphingosine/ceramid and spingomyelin phospholipid species can help diagnose Reverse Lipid Transport insufficiency, identify responders to treatment, assess treatment effect and predict treatment outcome with regard to preventing CVD events.

Results Study 2

Phosphatidyl Choline Phospholipids (PCs): serum concentrations of PC 36:0 and 36:1 were lowered by peptide treatment in C57B6 mice. The decreases in serum concentrations followed a dose-response pattern. For example 36:0 was lowered from 0.028 (0.003) ug/mL in control to 0.015 (0.004) in 101 mg/kg/48 h group to 0.010 (0.001) ug/mL in 101 mg/kg/24 h group. The Y26 peptide at 101 mg/kg/48 h lowered 36:0 to 0.010 (0.001) suggesting a more powerful treatment effect than for AT5261.

Treatment effects by peptides on Lyso-Phosphatidyl Choline Phospholipids (LPCs) were statistically significant already at 5 m/kg/24 h with little additional effects at higher doses. Treatment with AT5261 significantly lowered serum concentrations of LPC 20:3 and LPC 22:5 while serum concentrations of shorter LPCs like 16:0 and LPC 18:1 increased significantly. Y26 treatment effects followed the same pattern of increasing concentrations of shorter LPCs and decreasing serum concentrations of longer LPCs.

Total Sphingolipids (SLs) were statistically significantly lowered by daily peptide treatment in C57B mice going from 551 ug/mL in control to 378 in 5 mg/kg/24 (p<0.05) to 341 in 51 mg/kg/24 h (p<0.01) while only non-significant decreases were seen when peptide was given alternate days. Of the individual SLs the serum lowering effects were most pronounced for DiHy SM 20:0 where a dose response pattern and dosing interval response was seen with decreases up to 33% (p=0.0001). The results show that peptide treatment in C57B6 mice has unique effects on phospholipid mobilization from peripheral tissue to plasma. This animal model can be used for expanding knowledge about RLT insufficiency and for modeling treatment effects in other mammals including humans.

Results Study 3

AT5251 5 mg/kg/48 treatment in apoE KO mice for 6 weeks increased PC40:7 with 33% (p<0.05). Also PC 36:4 increased significantly with 27% (p<0.05).

AT5261 5 mg/kg/48 h treatment for 6 weeks in apoE KO mice decreased LPC 20:3 by 13% (p<0.05). AT5261 5 mg/kg/48 h treatment for 6 weeks decreased LPC 20:5 with 10% (p=0.014).

AT5261 treatment for 6 weeks in apoE KO mice showed selective and significant lowerings of sphingosine 18:0 (So 18:0) with no significant effects on the other SL species. Both 5 mg/kg daily and alternate day treatments lowered So(18) with 39% (p<0.05 for both groups compared to control).

This So 18:0 decrease in plasma is notable in view of the apoE KO flow cessation model which showed that So 18:0 concentrations were 24% highly significantly reduced in the carotid artery. The results in apoE KO mice show that peptide treatment effects have unique effects on phospholipid mobilization from peripheral tissue to plasma where change for certain serum phospholipid concentrations correlate to change in arterial tissue of the same phospholipid. This animal model can be used for expanding knowledge about RLT insufficiency, create biomarkers and companion diagnostics, and for modeling treatment effects in other mammals including humans.

### Example 7

Gene expression change by treatment with AT5261 can be assessed using gene chip technology (Phalanx, Palo Alto, Calif.). The objective is to see if gene expression changes by peptide correlated to those of HDL and apoA-I which are used as positive control and vehicle control as negative control.

Transformed J774 cells and mouse primary macrophages and cholesterol loading protocols using acetylated LDL are used to create foam cell macrophages.

Assessment of gene expression in various cell types including macrophage is used to assess reverse cholesterol insufficiency, responders to treatment and to assess progress of treatment effect.

The methods described herein, e.g., for assessing plasma lipids, plasma inflammation proteins, and gene expression from cells and from living species can be used to diagnose, assess and predict reverse lipid transport state and effects by RLT treatment.

### Example 8

### AT5261 and Y26 Incubation with Cynomolgus Monkey Plasma Cause Conversion of Large HDL to Small Preβ-HDL

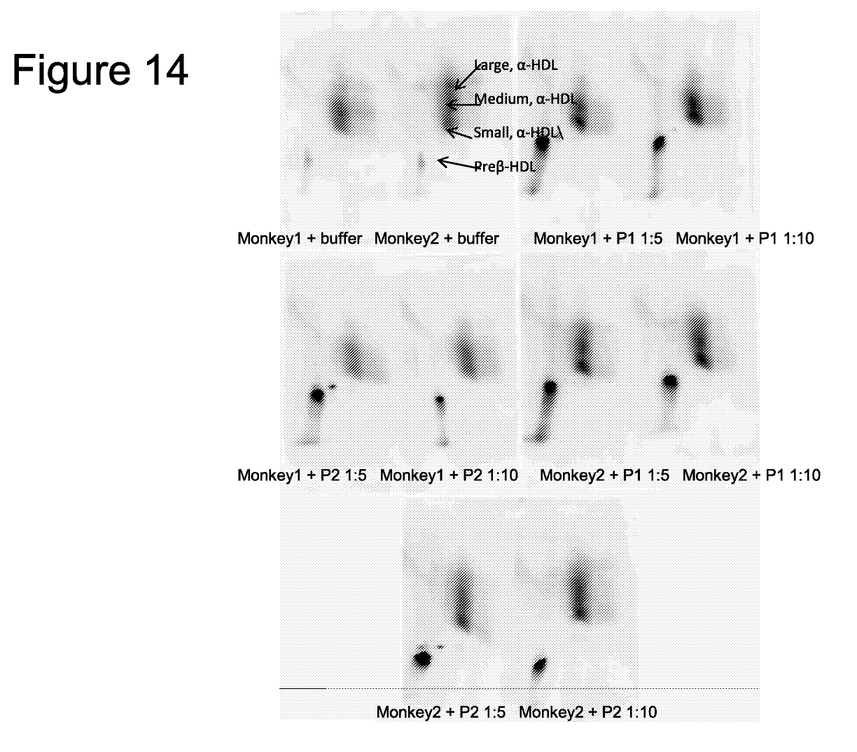

Each monkey sample was incubated with the same volume of peptide or PBS. AT5261 and Y26 Peptides were diluted 1:5 and 1:10 with PBS. Diluted peptide and plasma were mixed in 1:9 ratio and incubated for 5 minutes at room temperature before applying on the gel for approximate peptide to apoA-I mass concentration ratios of 0.1:1.0 and 0.05:1.0, respectively. Membranes were probed with anti monkey apoA-I. Large, medium and small size particles in the alpha front were selected because of the visible separation of these regions. Results are shown in FIG. 14.

Similar effects by peptides on HDL conversion were seen in human plasma, i.e. effect in non-human mammals can predict effects of peptide on preβ-HDL conversion in humans. This is important as preβ-HDL is nature's main ABCA1 transporter ligand facilitating cholesterol and phospholipid removal from for example macrophage foam cells, the main culprit in atherosclerosis disease. Table 6 shows the percent distribution of the HDL subfractions.

TABLE 6
Percent distribution of the HDL subfractions

| Inc condition | preb1 | preb x | larege a | medium a | small a | larege pre a | medium pre a | small pre a |
|---|---|---|---|---|---|---|---|---|
| M1 + buffer | 2.69 | 0 | 17.51 | 34.52 | 24.92 | 4.06 | 9.90 | 6.15 |
| M1 + peptide1 in 1:5 | 40.01 | 3.91 | 20.78 | 22.6 | 0.39 | 3.99 | 6.77 | 1.54 |
| M1 + peptide1 in 1:10 | 24.64 | 4.5 | 31.16 | 26.18 | 0.94 | 5.56 | 6.8 | 0.22 |
| M1 + peptide2 in 1:5 | 39.53 | 3.63 | 2.97 | 25.28 | 17.74 | 0.17 | 4.16 | 6.52 |
| M1 + peptide2 in 1:10 | 17.04 | 0 | 3.8 | 38.06 | 26.64 | 0.72 | 6.08 | 7.67 |
| M2 + buffer | 2.17 | 0 | 23.46 | 35.51 | 24.47 | 4.47 | 6.53 | 3.39 |
| M2 + peptide1 in 1:5 | 35.76 | 0.12 | 10.2 | 25.92 | 22.72 | 1.18 | 1.88 | 2.21 |
| M2 + peptide1 in 1:10 | 27.63 | 0.09 | 12.77 | 27.44 | 25.87 | 0.85 | 2.28 | 3.07 |
| M2 + peptide2 in 1:5 | 35.4 | 1.17 | 9.96 | 27.23 | 21.04 | 0.74 | 1.74 | 2.72 |
| M2 + peptide2 in 1:10 | 18.88 | 0 | 11.2 | 34.06 | 27.2 | 1.54 | 3.44 | 3.67 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of prognosing, diagnosing, and/or predicting a response to treatment of a condition associated with a deficiency in a reverse cholesterol transport (RCT) pathway in a subject comprising:
(a) providing a population of cells from the subject, wherein said population comprises at least one macrophage or a macrophage-like cell;
(b) contacting said population of cells with a modulator that specifically modulates a reverse cholesterol transporter pathway;
(c) assessing lipid efflux profile, mRNA expression, protein expression, protein activation level and/or a phenotype in said at least one macrophage or macrophage-like cell treated with said modulator or a medium comprising said cell;
(d) determining whether there is a deficiency in the reverse cholesterol transport pathway of the subject, wherein said determining is based in said assessing of lipid efflux profile, mRNA expression, protein expression, protein activation level and/or said phenotype in said at least one macrophage or macrophage-like cell or said medium comprising said cell; and
(e) prognosing, diagnosing, and/or predicting a response to treatment of said condition associated with a deficiency in a reverse cholesterol transport (RCT) pathway, wherein said prognosing, diagnosing, and/or predicting a response to treatment is based in said determining in step (d).

2. The method of claim 1 wherein the medium comprising said cell is tissue, organ, blood, serum, plasma, body fluid, or culture media.

3. A method of screening of compounds for treatment of a condition associated with reverse cholesterol transport deficiency and/or assessing risk of toxicity of a treatment of a condition associated with reverse cholesterol transport deficiency comprising:
(a) providing a macrophage or a macrophage-like cell, wherein said cell is contacted with a reverse cholesterol transport modulator;
(b) contacting the macrophage or macrophage-like cell with one or more compounds, wherein said one or more compounds are possible candidates for the treatment of a condition associated with reverse cholesterol transport deficiency, and/or wherein said one or more compounds are used in the treatment of a condition associated with reverse cholesterol transport deficiency;
(c) assessing lipid efflux profile, mRNA expression, protein expression, protein activation level and/or a phenotype in said macrophage or macrophage-like cell treated with said compound; and
(d) selecting said one or more compounds for treatment of said condition associated with reverse cholesterol transport deficiency and/or determining toxicity of a treatment of said condition associated with reverse cholesterol transport deficiency, wherein said selecting and/or said determining are based in said assessing from step (c).

4. The method of claim 1, wherein the condition associated with reverse cholesterol transport deficiency is a cardiovascular disease.

5. The method of claim 4, wherein the cardiovascular disease is atherosclerosis.

6. The method of claim 1, wherein the condition associated with reverse cholesterol transport deficiency is a neurological disease.

7. The method of claim 6, wherein the neurological disease is Alzheimer's disease.

8. The method of claim 1, wherein the modulator is a peptide or a peptide complex.

9. The method of claim 1, wherein said modulator modulates a reverse cholesterol transporter.

10. The method of claim 9 wherein said reverse cholesterol transporter is ABCA-1 or ABCG-1.

11. The method of claim 1, wherein the modulator is a peptide that modulates ABCA-1 or ABCG-1.

12. The method of claim 1, wherein the macrophage-like cell is a monocyte, or a foam cell.

13. The method of claim 3 wherein the macrophage-like cell is a monocyte, a foam cell, or a recombinant macrophage cell line.

14. The method claim 1, wherein the subject is a human.

15. The method of claim 1, wherein assessing the lipid efflux profile comprises measuring cholesterol, sphingosine, ceramide, sphingomyelin and triglyceride levels.

16. The method of claim 1, wherein the modulator modulates more than one RCT pathways.

17. The method of claim 1, wherein the subject is prognosed, diagnosed, and/or a response to treatment is predicted if there is a change in the lipid efflux profile, mRNA expression, protein expression, protein activation level and/or a phenotype as compared to that of a control cell.

18. The method of claim 1, further comprising comparing the lipid efflux profile, mRNA expression, protein expression, and/or protein activation level to a predetermined threshold value.

19. A method comprising prognosing, diagnosing, and/or predicting a response to treatment of a condition associated with a deficiency in a reverse cholesterol transport (RCT) pathway in a subject the method comprising
(a) administering a subject with a modulator that specifically modulates a reverse cholesterol transporter pathway
(b) assessing lipid efflux profile, mRNA expression, protein expression, protein activation level and/or a phenotype in at least one macrophage or macrophage-like cell from said subject or a medium comprising said cell;
(c) determining whether there is a deficiency in the reverse cholesterol transport pathway of the subject, wherein said determining is based in said assessing of lipid efflux profile, mRNA expression, protein expression, protein activation level and/or said phenotype in said at least one macrophage or macrophage-like cell or said medium comprising said cell; and
(d) prognosing, diagnosing, and/or predicting a response to treatment of said condition associated with a deficiency in a reverse cholesterol transport (RCT) pathway, wherein said prognosing, diagnosing, and/or predicting a response to treatment is based in said determining in step (c).

20. A method of prognosing, diagnosing, and/or predicting a response to treatment of a condition associated with a deficiency in a reverse cholesterol transport (RCT) pathway in a subject the method comprising
(a) administering a subject with a modulator that specifically modulates a reverse cholesterol transporter pathway
(b) assessing the mobilization of a biomarker from tissue to plasma in said subject; and
(c) prognosing, diagnosing, and/or predicting a response to treatment of said condition associated with a deficiency in a reverse cholesterol transport (RCT) pathway, wherein said prognosing, diagnosing, and/or predicting a response to treatment is based in said assessing in step (b).

21. The method of claim 19, wherein the condition associated with reverse cholesterol transport deficiency is a cardiovascular disease.

22. The method of claim 21, wherein the cardiovascular disease is atherosclerosis.

23. The method of claim 19, wherein the condition associated with reverse cholesterol transport deficiency is a neurological disease.

24. The method of claim 23, wherein the neurological disease is Alzheimer's disease.

25. The method of claim 19, wherein the modulator is a peptide or a peptide complex.

26. The method of claim 19, wherein said modulator modulates a reverse cholesterol transporter.

27. The method of claim 26 wherein said reverse cholesterol transporter is ABCA-1 or ABCG-1.

28. The method of claim 19, wherein the modulator is a peptide that modulates ABCA-1 or ABCG-1.

29. The method of claim 19, wherein the macrophage-like cell is a monocyte, or a foam cell.

30. The method of claim 19, wherein the subject is a human.

31. The method of claim 19, wherein the lipid efflux profile comprises cholesterol, sphingosine, ceramide, sphingomyelin and triglyceride levels.

32. The method of claim 19, wherein the modulator modulates more than one RCT pathways.

33. The method of claim 19, further comprising comparing the lipid efflux profile, mRNA expression, protein expression, and/or protein activation level to a predetermined threshold value.

34. The method of claim 1, wherein assessing lipid efflux profile comprises measuring the conversion of α-mobility HDL particles to pre-β1-HDL.

35. The method of claim 1, comprising measuring proteins selected from the group consisting of CRP, Fibrinogen, Haptoglobin, IL-18, SAP (serum amyloid P component), Rantes, TIMP-1, VCAM-1, MIP-1beta, MPO, VEGF-alpha and IL-7.

36. The method of claim 1, comprising measuring proteins involve in inflammation.


SEQUENCE LISTING (48 sequences)

SEQ ID NO: 1 - protein, 33 aa, Artificial Sequence
  Description of Artificial Sequence Synthetic polypeptide
PALEDLRQGLLPVLESFCVKFLSALEEYTKKLN

SEQ ID NO: 2 - protein, 26 aa, Artificial Sequence
  Description of Artificial Sequence Synthetic peptide
PVLESFKVSFLSALEEYKTKLESALN

SEQ ID NO: 3 - protein, 33 aa, Artificial Sequence
  Description of Artificial Sequence Synthetic polypeptide
QQARGWVTDGFSSLKDYWSTVKDKFSEFWDLDP

SEQ ID NO: 4 - protein, 55 aa, Artificial Sequence
  Description of Artificial Sequence Synthetic polypeptide
ARMEEMGSRTRDRLDEVKEQVAEVRAKLEEQAQQIRLQAEAFQARLKSWFEPLVE

SEQ ID NO: 5 - protein, 29 aa, Artificial Sequence
  Description of Artificial Sequence Synthetic peptide
DMQRQWAGLVEKVQAAVGTSAAPVPSDNH

SEQ ID NO: 6 - protein, 33 aa, Artificial Sequence
  Description of Artificial Sequence Synthetic polypeptide
ARMEEMGSRTRDRLDEVKEQVAEVRAKLEEQAQ

SEQ ID NO: 7 - protein, 22 aa, Artificial Sequence
  Description of Artificial Sequence Synthetic peptide
ARMEEMGSRTRDRLDEVKEQVA

SEQ ID NO: 8 - protein, 33 aa, Artificial Sequence
  Description of Artificial Sequence Synthetic polypeptide
EVRAKLEEQAQQIRLQAEAFQARLKSWFEPVLE

SEQ ID NO: 9 - protein, 33 aa, Artificial Sequence
  Description of Artificial Sequence Synthetic polypeptide
PLVEDMQRQWAGLVEKVQAAVGTSAAPVPSDNH

SEQ ID NO: 10 - protein, 26 aa, Artificial Sequence
  Description of Artificial Sequence Synthetic peptide
EVRAKLEEWFQQIRLQAEEFQARLKS

SEQ ID NO: 11 - protein, 33 aa, Artificial Sequence
  Description of Artificial Sequence Synthetic polypeptide
PFATELHERLAKDSEKLKEEIGKELEELRARLL

SEQ ID NO: 12 - protein, 25 aa, Artificial Sequence
  Description of Artificial Sequence Synthetic peptide
ELHERLAKDSEKLKEEIGKELEELR

SEQ ID NO: 13 - protein, 44 aa, Artificial Sequence
  Description of Artificial Sequence Synthetic polypeptide
PHADELKAKIDQNVEELKGRLTPYADEFKVKIDQTVEELRRSLA

SEQ ID NO: 14 - protein, 22 aa, Artificial Sequence
  Description of Artificial Sequence Synthetic peptide
PHADELKAKIDQNVEELKGRLT

SEQ ID NO: 15 - protein, 22 aa, Artificial Sequence
  Description of Artificial Sequence Synthetic peptide
PYADEFKVKIDQTVEELRRSLA

SEQ ID NO: 16 - protein, 44 aa, Artificial Sequence
  Description of Artificial Sequence Synthetic polypeptide
PYADEFKVKIDQTVEELRRSLAPYAQDTQEKLNHQLEGLTFQMK

SEQ ID NO: 17 - protein, 22 aa, Artificial Sequence
  Description of Artificial Sequence Synthetic peptide
PYAQDTQEKLNHQLEGLTFQMK

SEQ ID NO: 18 - protein, 44 aa, Artificial Sequence
  Description of Artificial Sequence Synthetic polypeptide
PYAQDTQEKLNHQLEGLTFQMKKNAEELKARISASAEELRQRLA

SEQ ID NO: 19 - protein, 22 aa, Artificial Sequence
  Description of Artificial Sequence Synthetic peptide
KNAEELKARISASAEELRQRLA

SEQ ID NO: 20 - protein, 22 aa, Artificial Sequence
  Description of Artificial Sequence Synthetic peptide
PYADQLRTQVNTQAEQLRRQLT

SEQ ID NO: 21 - protein, 22 aa, Artificial Sequence
  Description of Artificial Sequence Synthetic peptide
PLAQRMERVLRENADSLQASLR

SEQ ID NO: 22 - protein, 33 aa, Artificial Sequence
  Description of Artificial Sequence Synthetic polypeptide
LISRIKQSELSAKMREWFSETFQKVKEKLKIDS

SEQ ID NO: 23 - protein, 22 aa, Artificial Sequence
  Description of Artificial Sequence Synthetic peptide
SALDKLKEFGNTLEDKARELIS

SEQ ID NO: 24 - protein, 25 aa, Artificial Sequence
  Description of Artificial Sequence Synthetic peptide
IKQSELSAKMREWFSETFQKVKEKL

SEQ ID NO: 25 - protein, 31 aa, Artificial Sequence
  Description of Artificial Sequence Synthetic polypeptide
PTFLTQVKESLSSYWESAKTAAQNLYEKTYL

SEQ ID NO: 26 - protein, 25 aa, Artificial Sequence
  Description of Artificial Sequence Synthetic peptide
TQVKESLSSYWESAKTAAQNLYEKT

SEQ ID NO: 27 - protein, 26 aa, Artificial Sequence
  Description of Artificial Sequence Synthetic peptide
PAVDEKLRDLYSKSTAAMSTYTGIFT

SEQ ID NO: 28 - protein, 33 aa, Artificial Sequence; identical to SEQ ID NO: 3
  Description of Artificial Sequence Synthetic polypeptide

SEQ ID NO: 29 - protein, 25 aa, Artificial Sequence
  Description of Artificial Sequence Synthetic peptide
DGFSSLKDYWSTVKDKFSEFWDLDP

SEQ ID NO: 30 - protein, 51 aa, Artificial Sequence
  Description of Artificial Sequence Synthetic polypeptide
QAKEPCVESLVSQYFQTVTDYGKDLMEKVKSPELQAEAKSYFEKSKEQLTP

SEQ ID NO: 31 - protein, 28 aa, Artificial Sequence
  Description of Artificial Sequence Synthetic peptide
PCVESLVSQYFQTVTDYGKDLMEKVKSP

SEQ ID NO: 32 - protein, 36 aa, Artificial Sequence
  Description of Artificial Sequence Synthetic polypeptide
RSFFSFLGEAFDGARDMWRAYSDMREANYIGSDKYF

SEQ ID NO: 33 - protein, 34 aa, Artificial Sequence
  Description of Artificial Sequence Synthetic polypeptide
RSFFSFLGEAFDGARDMWRAYSDMREANYIGSDK

SEQ ID NO: 34 - protein, 26 aa, Artificial Sequence
  Description of Artificial Sequence Synthetic peptide
SFLGEAEFDGARDMWRAYSDMREANY

SEQ ID NO: 35 - protein, 26 aa, Artificial Sequence
  Description of Artificial Sequence Synthetic peptide
WAAEVISNARENIQRLTGHGAEDSLA

SEQ ID NO: 36 - protein, 33 aa, Artificial Sequence
  Description of Artificial Sequence Synthetic polypeptide
PALEDLRQGLLPVLESFKVSFLSALEEYTKKLN

SEQ ID NO: 37 - protein, 33 aa, Artificial Sequence
  Description of Artificial Sequence Synthetic polypeptide
LKLLDNWDSVTSTFSKLREQLGPALEDLRQGLL

SEQ ID NO: 38 - protein, 44 aa, Artificial Sequence
  Description of Artificial Sequence Synthetic polypeptide
ARLAEYHAKATEHLSTLSEKAKPVLESFKVSFLSALEEYTKKLN

SEQ ID NO: 39 - protein, 44 aa, Artificial Sequence
  Description of Artificial Sequence Synthetic polypeptide
PYSDELRQRLAARLEALKENGGPVLESFKVSFLSALEEYTKKLN

SEQ ID NO: 40 - protein, 44 aa, Artificial Sequence
  Description of Artificial Sequence Synthetic polypeptide
PLGEEMRDRARAHVDALRTHLAPVLESFKVSFLSALEEYTKKLN

SEQ ID NO: 41 - protein, 33 aa, Artificial Sequence
  Description of Artificial Sequence Synthetic polypeptide
PALEDLRQGLLLKLLDNWDSVTSTFSKLREQLG

SEQ ID NO: 42 - protein, 18 aa, Artificial Sequence
  Description of Artificial Sequence Synthetic peptide
DWFKAFYDKVAEKFKEAF

SEQ ID NO: 43 - protein, 18 aa, Artificial Sequence
  Description of Artificial Sequence Synthetic peptide
DWLKAFYDKVAEKLKEAF

SEQ ID NO: 44 - protein, 26 aa, Artificial Sequence
  Description of Artificial Sequence Synthetic peptide
EVRSKLEEWFAAFREFAEEFLARLKS

SEQ ID NO: 45 - protein, 26 aa, Artificial Sequence
  Description of Artificial Sequence Synthetic peptide
EVRSKLEEWFAAFREFFEEFLARLKS

SEQ ID NO: 46 - protein, 26 aa, Artificial Sequence
  Description of Artificial Sequence Synthetic peptide
EFRSKLEEWFAAFREFAEEFLARLKS

SEQ ID NO: 47 - protein, 44 aa, Artificial Sequence
  Description of Artificial Sequence Synthetic polypeptide
LKLLDNWDSVTSTFSKLREQLGPVTQEFWDNLEKETEGLRQEMS

SEQ ID NO: 48 - protein, 26 aa, Artificial Sequence
  Description of Artificial Sequence Synthetic peptide
EFRSKLEEWFAAFREFFEEFLARLKS